(19) United States
(12) Patent Application Publication
(10) Pub. No.: US 2025/0054091 A1
(43) Pub. Date: Feb. 13, 2025

(54) IMPROVEMENTS IN IMAGING DEVICES AND METHODS FOR MULTIPLE IMAGE ACQUISITION

(71) Applicant: Australian Lung Health Initiative Pty Ltd, Carlton (AU)

(72) Inventors: Andreas Fouras, Woodland Hills, CA (US); Jonathan Dusting, Carlton (AU)

(21) Appl. No.: 18/721,150

(22) PCT Filed: Dec. 22, 2022
(86) PCT No.: PCT/AU2022/051564

§ 371 Date: Jun. 17, 2024

(30) Foreign Application Priority Data
Dec. 22, 2021 (AU) 2021904205

(51) Int. Cl. G06T 1/00; G06T 7/00; G06T 7/20

(52) U.S. Cl. CPC G06T 1/0007; G06T 7/0012; G06T 7/20; G06T 2207/10088; G06T 2207/10116; G06T 2207/10132

(57) ABSTRACT

An imaging device and a method using the imaging device for acquiring in vivo images of a region of a subject's body. The imaging device comprises at least two energy sources, and at least two energy detectors for detecting energy from the at least two energy sources passing through the region of the subject's body. The imaging device also comprises a motion assembly configured to achieve relative movement between a source-detector pair comprising at least one of the energy source and energy detectors, and the subject, and a controller for operating the energy sources and detectors while stationary, to acquire a time series of in vivo images of the region of the subject's body, and operating the motion assembly and the source-detector pair to achieve relative movement therebetween to obtain a second set of images at each of a plurality of image angles in a plane of the source-detector pair.

This application claims priority from Australian Provisional Patent Application No. 2021904205 filed on 22 Dec. 2021, the contents of which are to be taken as incorporated herein by this reference.

### TECHNICAL FIELD

The present disclosure relates to an imaging device and method for acquiring in vivo images of a region of a human or animal subject's body. It also relates particularly but not exclusively to obtaining two images sets that can be used complimentarily e.g. for determining functional characteristics of an organ, such as the lungs or heart of the subject.

### BACKGROUND OF INVENTION

Current imaging modalities such as X-ray, Computed Tomography (CT) imaging and Magnetic Resonance Imaging (MRI) provide methods to examine the structure and function of organs of a patient, such as the lungs, heart and brain. However, structural lung change often arises after disease establishment, eliminating the possibility of disease-prevention treatments (e.g., in early cystic fibrosis). While high-resolution CT imaging can provide excellent structural detail, it is costly and the relatively high levels of radiation exposure (a high-resolution CT is often equivalent to 70 chest X-rays) are of concern. Due to ionizing radiation dose, use of X-ray based techniques (especially CT) for detection and treatment of various diseases, including acute respiratory disease, is severely restricted for vulnerable patients, such as infants and children who are more susceptible to tissue damage due to radiation. Furthermore, the inherent measurement limitations also severely restrict evidence-based detection and treatment of acute respiratory disease across all ages of patients.

XV technology developed by 4DMedical has offered a breakthrough in clinical lung function assessment. The XV technology is disclosed in patent applications published as WO 2011/032210 A1 and WO 2015/157799 A1. The current XV technique uniquely combines X-ray imaging with proprietary flow velocimetry algorithms to measure motion in all locations of the lung in fine spatial and temporal detail, enabling regional lung function measurements throughout the respiratory cycle, at every location within the lung. This approach enables detection of even subtle functional losses well before lung structure is irreversibly affected by disease, meaning that treatment may be applied early, when it has the greatest impact and the best chance of success.

Current XV technology is used in clinical applications via a Software as a Service (Saas) model, whereby scans of the patient's lungs are acquired using existing fluoroscopic X-ray equipment. The scans are then processed using software algorithms, via a cloud-based server, to provide functional imaging analysis of the patient's lungs over time. However, the accuracy and quality of the XV analysis is limited by the images able to be acquired using existing medical scanners which require patients to remain still and breathe in a controlled fashion during scanning.

This restricts access to many patient groups, including young children, the elderly, and patients with language, hearing or cognitive impairment, who are unable to be readily scanned due to positioning issues within the scanner and/or the inability to follow instructions for the scanning to be completed.

Computed Tomography (CT) scanners are commonly used to acquire cross-sectional images of a subject's body. Typical CT scanner arrangements employ a ring or c-shaped arm on which one energy source and typically one detector or detector array are mounted for rotation around the subject's body. Multiple images are acquired through X-ray measurements taken from different angles as the ring or c-shaped arm rotates which are used to produce cross-sectional images of the subject's body. A disadvantage of existing medical scanners, such as CT scanners, is that a large scanner is typically required for rotation around the subject's body to acquire images at different angles.

Furthermore, existing medical scanners, such as CT scanners, often employ X-rays which result in a high burden of X-ray radiation for the subject when multiple images are acquired at different angles for in vivo imaging. It would be desirable to reduce the X-ray dosage by shortening the operating time of the energy source and detector or detector array to acquire the images. Reducing the x-ray dosage is particularly beneficial to vulnerable patient groups, such as infants and children, who are more susceptible to tissue damage due to radiation.

FIGS. 1 and 2 illustrate an example of a system 10 for imaging a region 230 of a subject's body 210. System 10 includes three energy sources 11 and three detectors 12 spatially positioned in a common plane and located on a common arc 14 around the subject's body 210. The energy sources 11 and detectors 12 are stationary during scanning, adopting a fixed position in the system 10, in contrast to CT scanners with the rotating ring or c-shaped arm. The subject 200 may be positioned on a tray or bed 18 during imaging as shown. The spatial arrangement of the energy sources 11 and detectors 12 enables three imaging angles through the region 230 of the subject's body 210 to be captured during imaging as indicated by the imaging beams 16. FIG. 2 is a plan view of the system 10 of FIG. 1 omitting the detectors 12 for clarity and showing that the common plane with common arc 14 may be a transverse plane through the subject's body 210.

While the system 10 can capture multiple imaging angles, it requires the energy sources 11 and detectors 12 to be sufficiently spaced around the subject's body 210 in order to obtain enough imaging data for optimising image acquisition, such as for providing dynamic in vivo imaging capability. The energy sources 11 and detectors 12 of the system 10 shown in FIGS. 1 and 2 are equally spaced circumferentially around the subject's body 210 across a 360 degree angle. Similar to CT scanners, this arrangement would necessitate providing a large scanning device to acquire images at different angles where the stationary energy sources and detectors surround the patient's body.

Another disadvantage of existing medical scanners, such as CT scanners and the system 10 of FIGS. 1 and 2, is that the patient is often positioned in a patient tray or bed 18 in the scanner in a supine position. For dynamic imaging of the subject's lungs, the patient is required to remain still and breathe in a controlled fashion during scanning. This restricts access of the imaging technology to many patient groups, including young children, the elderly, and patients with language, hearing or cognitive impairment, who are unable to be readily scanned due to positioning issues within the scanners and/or the inability to follow instructions for the scanning to be completed.

International patent application PCT/AU2021/050669 filed 25 Jun. 2021 describes an imaging device and method for obtaining a time sequence of images for use in construction of a motion field or determination of motion measurements indicative of e.g. lung function. However, clinical value of these measurements can be limited in the absence of contextual information such as e.g. the geometric structure or shape of the organ and its position in the thoracic cavity.

It would be desirable to provide an imaging device and/or imaging method that ameliorates and/or overcomes one or more problems or inconveniences of the prior art.

A reference herein to a patent document or any other matter identified as prior art, is not to be taken as an admission that the document or other matter was known or that the information it contains was part of the common general knowledge as at the priority date of any of the claims.

### SUMMARY OF INVENTION

Viewed from one aspect, the present disclosure provides an imaging device for acquiring in vivo images of a region of a subject's body, the imaging device comprising: at least two energy sources; at least two energy detectors for detecting energy from the at least two energy sources passing through the region of the subject's body located between the energy sources and the energy detectors, wherein a first source-detector pair is located in a first plane, and a second source-detector pair is located in a second plane, wherein the first plane and the second plane intersect through the region of the subject's body to be imaged; a motion assembly configured to achieve relative movement between at least one of the source-detector pairs and the subject; and a controller configured to: operate the energy sources and detectors while stationary, to acquire a time series of in vivo images of the region of the subject's body; and operate the motion assembly and at least one source-detector pair to achieve relative movement between the at least one source-detector pair and the subject to obtain a second set of images at each of a plurality of image angles in the plane of the source-detector pair.

In some embodiments, the imaging device further comprises a third source-detector pair located in the first plane, and a fourth source-detector pair located in the second plane. The imaging device may optionally exclude the fourth source-detector pair.

In some embodiments, the time series of in vivo images are used to generate a motion measurement of the region and the second set of images are used to construct a geometric structure image of the region. Preferably, the geometric structure image is a three-dimensional geometric structure image of the region.

In some embodiments, the second set of images are obtained at image angles covering an arc of no more than about 180 degrees, about 160 degrees, about 140 degrees, about 120 degrees, about 100 degrees, and about 80 degrees in the plane of the source-detector pair.

In some embodiments, the controller is configured to operate the at least one source-detector pair and the motion assembly to obtain the second set of images such that it acquires less than about 200 images, or less than about 120 images, such as less than about 100 images, or less than about 80 images, possibly less than about 50 images, or less than about 40 images, such as about or less than about 20 images, such as about 10 images.

In some embodiments, the controller is configured to operate the motion assembly for continuous relative movement while the source-detector pair obtains the second set of images at each of the plurality of image angles. In other embodiments, the controller is configured to operate the motion assembly for discrete relative movements between operation of the at least one source-detector pair to obtain an image at each of the plurality of image angles.

In some embodiments, the motion assembly comprises a source frame supporting the source of the at least one source-detector pair, wherein the controller is configured to control linear motion of the source along the source frame to obtain the second set of images. The source frame may comprise a straight frame or it may comprise a curved frame.

In some embodiments, the motion assembly comprises a detector frame supporting the detector of the at least one source-detector pair, wherein the controller is configured to control linear motion of the detector along the detector frame to obtain the second set of images. The detector frame may comprise a straight frame or it may comprise a curved frame.

In some embodiments, the controller is configured to control movement of the source on the source frame and the detector on the detector frame in synchrony.

In some embodiments, the imaging device further comprises a supplementary source on the source frame, and the controller is configured to control linear motion of the supplementary source along the source frame to obtain the second set of images. Provision of the supplementary source may reduce the distance required to be travelled by any source to obtain the second set of images at each of the plurality of image angles. The controller may be configured to control movement of the supplementary source in synchrony with the source of the source-detector pair e.g. coordinating movement so as to avoid collision.

In some embodiments, the imaging device further comprises a supplementary detector on the detector frame, and the controller is configured to control linear motion of the supplementary detector along the detector frame to obtain the second set of images. Provision of the supplementary detector may reduce the distance required to be travelled by any source to obtain the second set of images at each of the plurality of image angles. The controller may be configured to control movement of the supplementary detector in synchrony with the detector of the source-detector pair, e.g. coordinating movement so as to avoid collision.

In some embodiments, the source frame provides one or more couplings for angular and linear motion of any source coupled to the source frame, and the controller is configured to control angular and linear motion of any source coupled to the source frame.

In some embodiments, the detector frame provides one or more couplings for angular and linear motion of any detector coupled to the detector frame, and wherein the controller is configured to control angular and linear motion of any detector coupled to the detector frame.

The angular and linear motion of any source or detector may be performed under control of the controller substantially simultaneously with linear and angular motions occurring together, or it may be performed stepwise with linear movements occurring separately from angular movements.

The source frame and the detector frame may be oriented vertically, horizontally, or in a transverse direction relative to the horizonal and vertical directions.

In some embodiments, two or more source-detector pairs are movable by the motion assembly to obtain the second set of images.

In some embodiments, the imaging device further comprises one or more positional sensors configured to sense one or both of linear and angular position of one or more sources and/or one or more detectors of the device for input to the controller.

In some embodiments, the motion assembly comprises a movable platform operable by the controller to control movement of the subject relative to the one or more source-detector pairs to obtain the second set of images. The movable platform may be operable to rotate the subject around a cranio-caudal axis between the sources and the detectors. In other embodiments, the movable platform may be operable to rotate the subject around a dorso-ventral axis (e.g. in a frontal plane).

In some embodiments, the imaging device further comprises an auxiliary energy detector for detecting energy from one of the energy sources providing a source-auxiliary detector pair. The motion assembly may be further configured to achieve relative movement between the source-auxiliary detector pair and the subject, and the controller may be further configured to operate the motion assembly and the source-auxiliary detector pair to achieve relative movement between the source-auxiliary detector pair and the subject to obtain the second set of images at each of the plurality of image angles in a plane of the source-auxiliary detector pair.

The at least one source-detector pair and the source-auxiliary detector pair may be movable by the motion assembly to obtain the second set of images. Thus, two or three sets of source-detector pairs (e.g., including the source-auxiliary detector pair) may be used to obtain the second set of images.

Viewed from another aspect, the present disclosure provides an imaging device for acquiring in vivo images of a region of a subject's body, the imaging device comprising: at least two energy sources; at least two energy detectors for detecting energy from the at least two energy sources passing through the region of the subject's body located between the energy sources and the energy detectors, wherein a first source-detector pair is located in a first plane, and a second source-detector pair is located in a second plane, wherein the first plane and the second plane intersect through the region of the subject's body to be imaged; an auxiliary energy detector for detecting energy from one of the energy sources providing a source-auxiliary detector pair; a motion assembly configured to achieve relative movement between the source-auxiliary detector pair and the subject; and a controller configured to: operate the energy sources and detectors while stationary, to acquire a time series of in vivo images of the region of the subject's body; and operate the motion assembly and the source-auxiliary detector pair to achieve relative movement between the source-auxiliary detector pair and the subject to obtain a second set of images at each of a plurality of image angles in a plane of the source-auxiliary detector pair.

In some embodiments, the imaging device further comprises a third source-detector pair located in the first plane, and a fourth source-detector pair located in the second plane. The imaging device may optionally exclude the fourth source-detector pair.

In some embodiments, the time series of in vivo images are used to generate a motion of the region and the second set of images are used to construct a geometric structure image of the region. Preferably, the geometric structure image is a three-dimensional geometric structure image of the region.

In some embodiments, the second set of images are obtained at image angles covering an arc in the plane of the source-auxiliary detector pair of no more than one of: about 180 degrees, about 160 degrees, about 140 degrees, about 120 degrees; about 100 degrees; or about 80 degrees. The plane of the source-auxiliary detector pair may pass through the region of the subject's body to be imaged.

In some embodiments, the controller is configured to operate the source-auxiliary detector pair and the motion assembly to obtain the second set of images such that it acquires less than one of: about 200 images; about 120 images; about 100 images; about 80 images; about 50 images; about 40 images; about 20 images; or about 10 images.

In some embodiments, the controller is configured to operate the motion assembly for continuous relative movement while the source-auxiliary detector pair obtains the second set of images at each of plurality of image angles. In other embodiments, the controller is configured to operate the motion assembly for discrete relative movements between operation of the source-auxiliary detector pair to obtain an image at each of the plurality of image angles.

In some embodiments, the motion assembly comprises a source frame supporting the source of the source-auxiliary detector pair, wherein the controller is configured to control linear motion of the source along the source frame to obtain the second set of images. The source frame may comprise a straight frame or it may comprise a curved frame.

In some embodiments, the motion assembly comprises a detector frame supporting the auxiliary detector of the source-auxiliary detector pair, wherein the controller is configured to control linear motion of the detector along the detector frame to obtain the second set of images. The detector frame may comprise a straight frame or it may comprise a curved frame.

In some embodiments, the controller is configured to control movement of the source on the source frame and the auxiliary detector on the detector frame in synchrony.

In some embodiments, the imaging device further comprises a supplementary source on the source frame. The controller may be configured to control linear motion of the supplementary source along the source frame to obtain the second set of images. Provision of the supplementary source may reduce the distance required to be travelled by any source to obtain the second set of images at each of the plurality of image angles. The controller may be configured to control movement of the supplementary source in synchrony with the source of the source-auxiliary detector pair.

In some embodiments, the imaging device further comprises a supplementary detector on the detector frame. The controller may be configured to control linear motion of the supplementary detector along the detector frame to obtain the second set of images. The controller may be configured to control movement of the supplementary detector in synchrony with the auxiliary detector of the source-auxiliary detector pair.

In some embodiments, the source frame provides one or more couplings for angular and linear motion of any source coupled to the source frame, and the controller is configured to control angular and linear motion of any source coupled to the source frame.

In some embodiments, the detector frame provides one or more couplings for angular and linear motion of any detector coupled to the detector frame, and the controller is configured to control angular and linear motion of any detector coupled to the detector frame.

The source frame and the detector frame may be oriented vertically, horizontally, or in a transverse direction relative to the horizonal and vertical directions.

In some embodiments, the imaging device further comprises one or more positional sensors configured to sense one or both of linear and angular position of one or more sources and/or one or more detectors of the device for input to the controller.

In some embodiments, the motion assembly comprises a movable platform operable by the controller to control movement of the subject relative to the source-auxiliary detector pair to obtain the second set of images. The movable platform may be operable to rotate the subject around a cranio-caudal axis between the sources and the detectors. The movable platform may be operable to rotate the subject around a dorso-ventral axis.

In some embodiments, the motion assembly is further configured to achieve relative movement between at least one of the source-detector pairs and the subject. The controller may be further configured to operate the motion assembly and the at least one source-detector pair to achieve relative movement between the source-detector pair and the subject to obtain the second set of images at each of the plurality of image angles in the plane of the source-detector pair.

The source-auxiliary detector pair and the at least one source-detector pair may be movable by the motion assembly to obtain the second set of images. Thus, two or three sets of source-detector pairs (e.g., including the source-auxiliary detector pair) may be used to obtain the second set of images.

Viewed from another aspect, the present disclosure provides an imaging device for acquiring in vivo images of a region of a subject's body, the imaging device comprising: at least two energy sources; at least two energy detectors for detecting energy from the at least two energy sources passing through the region of the subject's body located between the energy sources and the energy detectors, wherein a first source-detector pair is located in a first plane, and a second source-detector pair is located in a second plane, wherein the first plane and the second plane intersect through the region of the subject's body to be imaged; a motion assembly configured to achieve relative movement between at least one source-detector pair comprising at least one of the energy sources and energy detectors, and the subject; and a controller configured to: operate the energy sources and detectors while stationary, to acquire a time series of in vivo images of the region of the subject's body; and operate the motion assembly and the at least one source-detector pair to achieve relative movement between the at least one source-detector pair and the subject to obtain a second set of images at each of a plurality of image angles in a plane of the source-detector pair.

In some embodiments, the imaging device further comprises a third source-detector pair located in the first plane, and a fourth source-detector pair located in the second plane. The imaging device may optionally exclude the fourth source-detector pair.

In some embodiments, the time series of in vivo images are used to generate a motion of the region and the second set of images are used to construct a geometric structure image of the region. Preferably, the geometric structure image is a three-dimensional geometric structure image of the region.

In some embodiments, the second set of images are obtained at image angles covering an arc in the plane of the at least one source-detector pair of no more than one of: about 180 degrees, about 160 degrees, about 140 degrees, about 120 degrees; about 100 degrees; or about 80 degrees.

In some embodiments, the controller is configured to operate the at least one source-detector pair and the motion assembly to obtain the second set of images such that it acquires less than one of: about 200 images; about 120 images; about 100 images; about 80 images; about 50 images; about 40 images; about 20 images; or about 10 images.

In some embodiments, the controller is configured to operate the motion assembly for continuous relative movement while the source-detector pair obtains the second set of images at each of plurality of image angles. In other embodiments, the controller is configured to operate the motion assembly for discrete relative movements between operation of the at least one source-detector pair to obtain an image at each of the plurality of image angles.

In some embodiments, the motion assembly comprises a source frame supporting the source of the at least one source-detector pair, wherein the controller is configured to control linear motion of the source along the source frame to obtain the second set of images. The source frame may comprise a straight frame or it may comprise a curved frame.

In some embodiments, the motion assembly comprises a detector frame supporting the detector of the at least one source-detector pair, wherein the controller is configured to control linear motion of the detector along the detector frame to obtain the second set of images. The detector frame may comprise a straight frame or it may comprise a curved frame.

In some embodiments, the controller is configured to control movement of the source on the source frame and the detector on the detector frame in synchrony.

In some embodiments, the imaging device further comprises a supplementary source on the source frame. The controller may be configured to control linear motion of the supplementary source along the source frame to obtain the second set of images. Provision of the supplementary source may reduce the distance required to be travelled by any source to obtain the second set of images at each of the plurality of image angles. The controller may be configured to control movement of the supplementary source in synchrony with the source of the source-detector pair.

In some embodiments, the imaging device further comprises a supplementary detector on the detector frame. The controller may be configured to control linear motion of the supplementary detector along the detector frame to obtain the second set of images. The controller is configured to control movement of the supplementary detector in synchrony with the detector of the source-detector pair.

In some embodiments, the source frame provides one or more couplings for angular and linear motion of any source coupled to the source frame, and the controller is configured to control angular and linear motion of any source coupled to the source frame.

In some embodiments, the detector frame provides one or more couplings for angular and linear motion of any detector coupled to the detector frame, and the controller is configured to control angular and linear motion of any detector coupled to the detector frame.

The source frame and the detector frame may be oriented vertically, horizontally, or in a transverse direction relative to the horizonal and vertical directions.

In some embodiments, two or more source-detector pairs are movable by the motion assembly to obtain the second set of images.

In some embodiments, the imaging device further comprises one or more positional sensors configured to sense one or both of linear and angular position of one or more sources and/or one or more detectors of the device for input to the controller.

In some embodiments, the motion assembly comprises a movable platform operable by the controller to control movement of the subject relative to the source-detector pair to obtain the second set of images. The movable platform may be operable to rotate the subject around a cranio-caudal axis between the sources and the detectors. The movable platform may be operable to rotate the subject around a dorso-ventral axis.

In some embodiments, the imaging device further comprises an auxiliary energy detector for detecting energy from one of the energy sources providing a source-auxiliary detector pair. The motion assembly may be configured to achieve relative movement between the source-auxiliary detector pair and the subject. The controller may be configured to operate the motion assembly and the source-auxiliary detector pair to achieve relative movement between the source-auxiliary detector pair and the subject to obtain the second set of images at each of the plurality of image angles in a plane of the source-auxiliary detector pair.

The source-auxiliary detector pair may be the only source-detector pair that is used by the imaging device to obtain the second set of images. In other embodiments, at least one of the source-detector pairs and the source-auxiliary detector pair may be movable by the motion assembly to obtain the second set of images. Thus, two or three sets of source-detector pairs (e.g., including the source-auxiliary detector pair) may be used to obtain the second set of images.

Viewed from another aspect, the present disclosure provides a method for acquiring in vivo images of a region of a subject's body, the method comprising the steps of: providing an imaging device according to any one of the previously described aspects of the disclosure; operating the controller to acquire the time series of in vivo images of the region of the subject's body simultaneously or substantially at the same time from each of the detectors; operating the controller to acquire the second set of in vivo images of the region of the subject's body for each of the plurality of angles; constructing, using a processor, a motion measurement based on the time series of images acquired from each of the detectors; constructing, using a processor, a geometric structure image based on the second set of images obtained from the detectors for each of the plurality of angles; and constructing, using a processor, a hybrid image in which the motion measurement and the geometric structure image are combined.

In some embodiments, the method comprises the step of, prior to operating the controller to acquire the images, positioning the subject in the imaging device between the energy sources and detectors.

The imaging device may be configured for use with one or more of x-ray imaging, ultrasound imaging, and magnetic resonance imaging (MRI). The x-ray imaging may include fluoroscopic imaging and/or computed tomographic x-ray velocity (CTXV) imaging.

The region of the subject's body to be imaged may include at least part of the lungs of the subject. The imaging device may image part of the lung or the whole lung. The imaging device may also image both lungs of the subject. The hybrid image may provide visible elements designating geometric features of the lungs.

Alternatively, the region to be imaged may include part or the whole of the heart or brain of the subject. The region to be imaged may include parts of the body other than organs, including tissues, such as abdominal tissues.

Ideally, the subject's breathing is not restricted or controlled during image acquisition. The imaging device may be configured to acquire the images while the subject is breathing and preferably of a full single breath of the subject.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1 is a perspective view of a system for imaging a region of a subject's body, where the system includes three energy sources and three detectors positioned in a common plane and located on a common arc around the subject's body which is located in a supine position on a tray during scanning.

FIG. 2 is a plan view of the system of FIG. 1 showing three energy sources in a common plane and located on a common arc around the subject's body, and omitting the detectors for clarity.

FIG. 3 is a plan view of an imaging device according to some embodiments, showing three energy sources spatially positioned around a subject's body in an approximately triangular-shaped or L-shaped configuration, where the subject's body is oriented in a supine position and the detectors have been omitted for clarity.

FIG. 4 is a plan view of another imaging device according to some embodiments, showing four energy sources spatially positioned around a subject's body in an approximately T-shaped configuration, where the subject's body is oriented in a supine position and the detectors have been omitted for clarity.

FIG. 5 is a perspective view of another imaging device according to some embodiments, showing four energy sources and four detectors each spatially positioned around a subject's body in an approximately diamond-shaped configuration, where the subject's body is oriented in an upright standing position in the scanner.

FIG. 6 is a perspective view of another imaging device according to some embodiments, showing four energy sources and four detectors each spatially positioned around a subject's body in an approximately square-shaped configuration, where the subject's body is oriented in an upright standing position in the scanner.

FIG. 7 is a perspective view of another imaging device according to some embodiments, showing four energy sources positioned in an exemplary source unit and four detectors positioned in an exemplary detector unit of the imaging device as shown in broken lines, the four energy sources and four detectors each being spatially positioned around a subject's body in an approximately diamond-shaped configuration, where the subject's body is oriented in an upright seated position in the scanner.

FIG. 8 is a perspective view of the imaging device of FIG. 7 excluding the exemplary detector unit and source unit for clarity.

FIG. 9 is a plan view of the imaging device of FIG. 7 excluding the exemplary detector unit and source unit for clarity.

FIG. 10 is a perspective view of another imaging device according to some embodiments of the disclosure, showing a similar arrangement to FIG. 7 except that two of the detectors are angled relative to the respective energy sources, and are co-planar and vertically oriented relative to one another.

FIG. 11 is a perspective view of the imaging device of FIG. 10 excluding the exemplary detector unit and source unit for clarity.

FIG. 12 is a plan view of the imaging device of FIG. 10 excluding the exemplary detector unit and source unit for clarity.

FIG. 13 is a schematic diagram showing components of an exemplary detector unit and source unit of the imaging device according to some embodiments of the disclosure.

FIG. 14 is a flow chart showing steps in a method for imaging according to some embodiments of the disclosure.

FIGS. 15A and 15B illustrate an imaging device with a motion assembly in two different positional configurations respectively according to an embodiment of the disclosure.

FIG. 16 illustrates an example of an imaging device with a motion assembly and a supplementary source, according to an embodiment of the disclosure.

FIG. 17 is a schematic illustration showing various arrangements of the source-detector pairs that may be used to obtain the second set of images according to various embodiments of the disclosure.

FIGS. 18 and 19 are perspective views of another imaging device with a motion assembly, according to some embodiments of the disclosure, where the subject is positioned on a tray and two sources for obtaining the second set of images are located in an arcuate source frame above the subject.

FIG. 20 is a perspective view of the imaging device of FIG. 18 excluding the exemplary detector unit and source unit, and the motor assembly for clarity.

FIG. 21 is a side view of the imaging device of FIGS. 18 and 19 showing transparent features including an auxiliary detector for obtaining the second set of images, according to some embodiments of the disclosure, where the auxiliary detector is in a disabled configuration.

FIGS. 22 to 24 are side views of the imaging device shown in FIG. 21 with the auxiliary detector shown in three different positions in an enabled configuration for obtaining the second set of images, the auxiliary detector being moveable beneath and parallel to the subject's body on the tray via the motor assembly and also synchronously with a source unit above in the arcuate source frame, adopting a location aligned near the subject's pelvis (FIG. 22), the subject's chest (FIG. 23) and the subject's neck (FIG. 24), according to some embodiments of the disclosure.

FIG. 25 is a schematic timing diagram illustrating operation of the source and detector to acquire a series of n images according to embodiments of the disclosure.

FIG. 26 is a schematic illustration exemplifying motion control by the controller according to various embodiments of the disclosure.

FIGS. 27A and 27B are schematic illustrations of control system architectures that may be utilised in various embodiments of the disclosure.

FIG. 28 is a flow chart showing steps in a method for obtaining a second set of images according to embodiments of the disclosure.

### DETAILED DESCRIPTION

Embodiments of the disclosure are discussed herein by reference to the drawings which are not to scale and are intended merely to assist with explanation. Reference herein to a subject may include a human or animal subject, or a human or animal patient on which medical procedures are performed and/or screening, monitoring and/or diagnosis of a disease or disorder is performed. In relation to animal patients, embodiments of the disclosure may also be suitable for veterinary applications. The terms subject and patient, and imaging device and scanner, respectively, are used interchangeably throughout the description and should be understood to represent the same feature of embodiments of the disclosure. Reference herein is also provided to anatomical planes of a subject's body, including the transverse or horizontal plane, the sagittal or vertical plane, and the coronal or frontal plane through the subject's body.

Preferably, the region to be imaged includes one or both lungs of the subject, or part of a lung of the subject. Alternatively, the region to be imaged may include part of or the whole of the heart or brain of the subject. Other organs or regions of the subject's body may also be suitable for functional imaging, such as those in which dynamic in vivo changes are detectable including changes in motion, location and/or size, during breathing or other physiological processes of the subject's body, as would be appreciated by a person skilled in the art.

The images acquired comprise a time sequence of in vivo images obtained using stationary source-detector pairs and a second set of images obtained during operation of a motion assembly. The acquisition of the time sequence of images is further described in PCT/AU2021/050669 filed 25 Jun. 2021 the entire disclosure of which is hereby incorporated herein by reference. The time series of images is ideally of the type suitable for XV processing in accordance with the techniques described in International Patent Application No. PCT/AU2010/001199 filed on 16 Sep. 2010 and published as WO 2011/032210 A1 on 24 Mar. 2011 filed in the name of Monash University, and International Patent Application No. PCT/AU2015/000219 filed on 14 Apr. 2015 and published as WO 2015/157799 A1 on 22 Oct. 2015 filed in the name of 4Dx Pty Ltd, the entire disclosures of both of which are incorporated herein by this reference. Thus, the images acquired may be processed using the XV technique described in those disclosures to provide a three-dimensional motion field of the region imaged, which preferably represents the three spatial dimensions over time of the region imaged. In the context of imaging of the lungs, this allows for motion of the lungs to be measured throughout the respiratory cycle, enabling evaluation of lung function at each region within the lung in fine spatial and temporal detail. Similar images may be obtained for other regions of the subject's body, including the heart or brain, or other organs or regions in which dynamic in vivo changes are detectable.

However, the ability to interpret the function of the lungs (or other organ or region of the body) may be improved by also obtaining a geometric structure image, preferably a 3D geometric structure image of the organ or region of interest. The present disclosure provides an imaging device and method that provides for acquisition of both a time series of images for obtaining a motion measurement of the region, and a second set of images for constructing a geometric structure image. These sets of images can be processed to produce a hybrid view of the region of interest, e.g. the lung, which provides functional information from the motion measurement, as well as anatomical context from the geometric structure image.

The imaging device may be suitable for X-ray imaging techniques, together with other imaging methods that do not involve the use of X-rays. In particular, the imaging device and method may be configured for one or more of x-ray imaging, ultrasound imaging, and magnetic resonance imaging (MRI). The imaging device and related method may be configured for use with static or dynamic x-ray imaging techniques. Dynamic x-ray imaging techniques may include fluoroscopic imaging and/or computed tomographic x-ray velocity (CTXV) imaging. The imaging device 100 and method 500 are preferably configured for fluoroscopic imaging.

For context, a description of the imaging device and method for obtaining images for construction of a geometric structure image will be preceded by a description of a prior imaging device and method that may be used for obtaining images for construction of a motion field or motion measurement.

### Obtaining Images for Motion Measurement

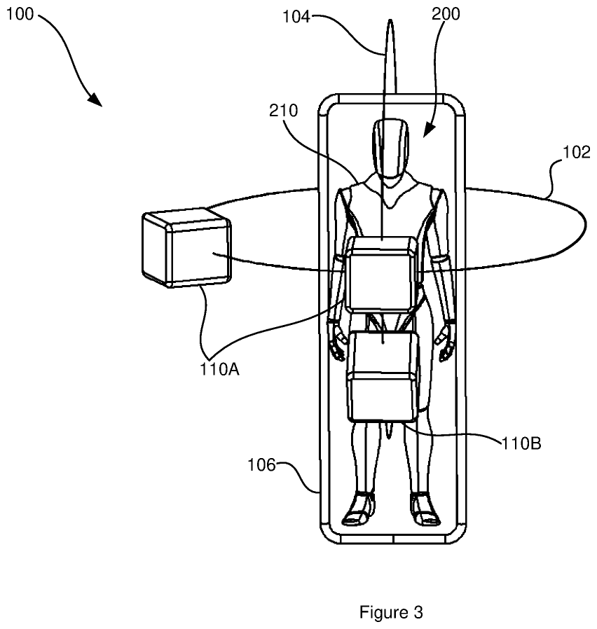

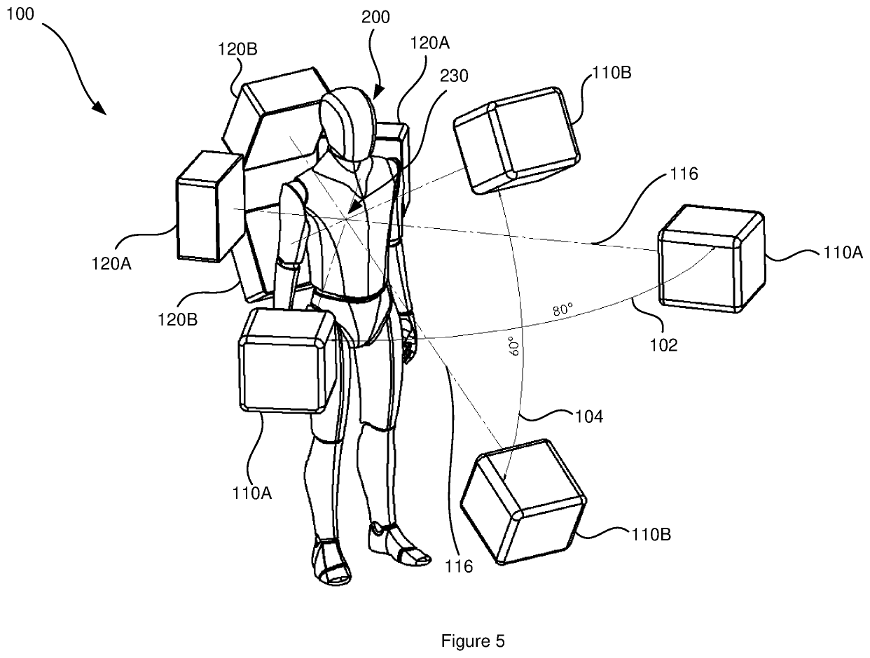

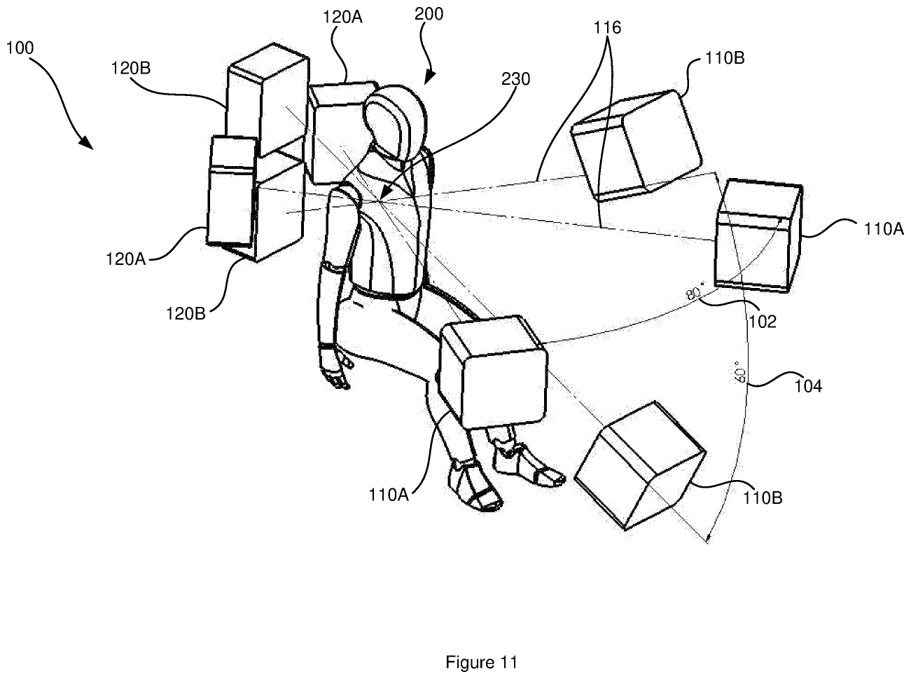

Disclosed herein is an imaging device 100 for acquiring a time series of in vivo images of a region 230 of a subject's body 210, as shown in the embodiments of FIGS. 3 to 13. The imaging device 100 includes at least three energy sources 110 (denoted as 110A, 110B) and at least three detectors 120 (denoted as 120A, 120B) for detecting energy from the at least three energy sources 110 passing through the region 230 of the subject's body 210 located between the energy sources 110 and detectors 120. At least two pairs of energy sources and detectors 110A, 120A are spatially positioned around the subject's body 210 in a first plane, and at least one pair of energy sources and detectors 110B, 120B is spatially positioned around the subject's body 210 in a second plane. The first plane and the second plane intersect through the region 230 of the subject's body 210 to be imaged (see also FIG. 5). The imaging device 100 also includes a controller 140 configured to operate the energy sources 110A, 110B and detectors 120A, 120B to acquire a time series of in vivo images of the region 230 of the subject's body 210. The energy sources 110 and detectors 120 are stationary during scanning, adopting a fixed position in the imaging device 100.

Preferably, the region 230 to be imaged may include at least part of a lung of the subject 200, and the duration of imaging may be based on a subject's single breath. Desirably, the imaging device 100 enables multiple time series of images to be acquired of either part or a single breath of the subject 200. This may include inspiration, expiration or both inspiration and expiration for a full breath. Preferably, the imaging device 100 enables multiple time series to be acquired of a full single breath of the subject 200.

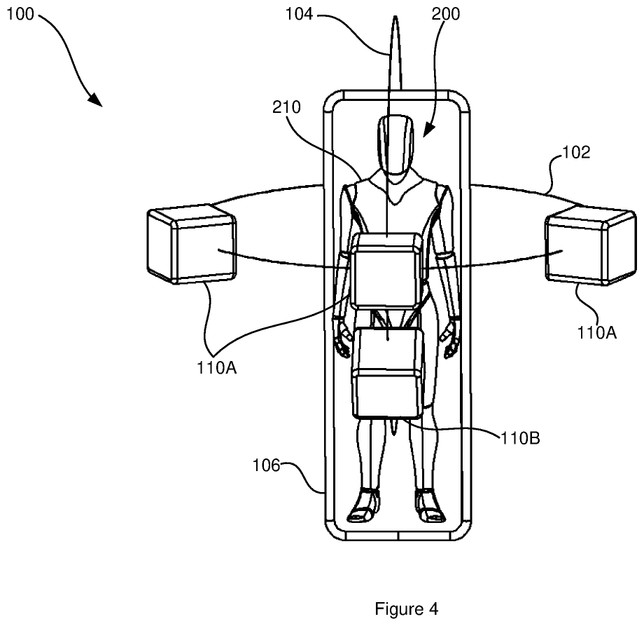

In some embodiments, the controller 140 is configured to acquire the images using at least three imaging angles through the region 230 of the subject's body 210. At least two imaging angles may be provided in the first plane through the subject's body 210, and at least one imaging angle may be provided in the second plane through the subject's body 210. The spatial arrangement and positioning of the pairs of energy sources and detectors to provide the at least three imaging angles will be discussed in more detail below in relation to the embodiment of FIG. 3. In the embodiments of FIGS. 4 to 12, the controller 140 is configured to acquire the images using at least four imaging angles through the region 230 of the subject's body 210, with at least two imaging angles being provided in each of the first and second planes through the subject's body 210.

Imaging device 10 typically includes at least three pairs of energy sources 110 and detectors 120 (see FIG. 3) or preferably, four pairs of energy sources 110 and detectors 120 (see FIGS. 4 to 12). This enables at least three, and preferably four, time series of in vivo images to be acquired during scanning. By acquiring a time series of images from multiple angles it is possible to provide dynamic imaging of the subject's body 210. In particular, embodiments of the disclosure may be suitable for functional imaging, such as those in which dynamic in vivo changes are detectable including changes in motion, location and/or size of organs or regions of the body, during breathing or other physiological processes of the subject's body 210, as would be appreciated by a person skilled in the art. This will be described in more detail in relation to method 300 and processing of the acquired images using XV techniques.

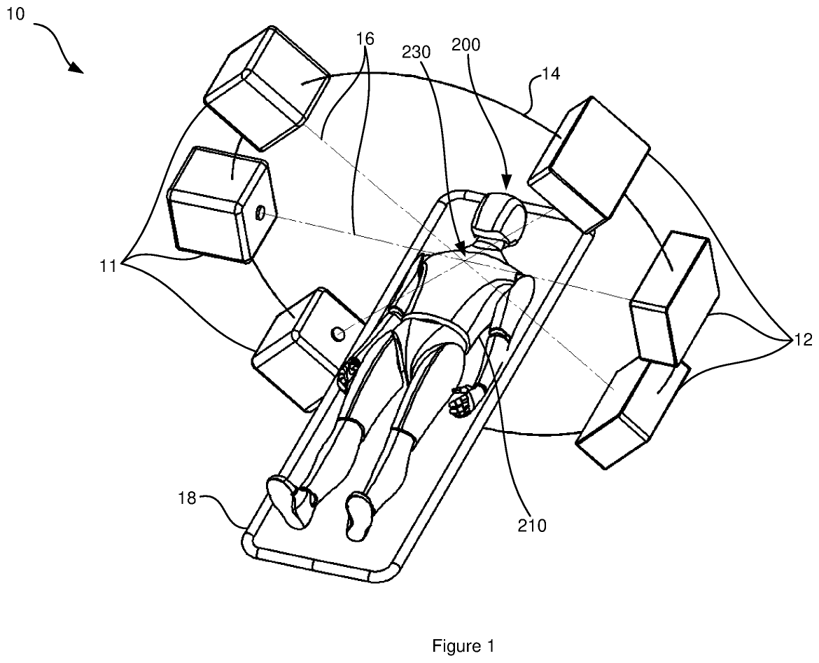

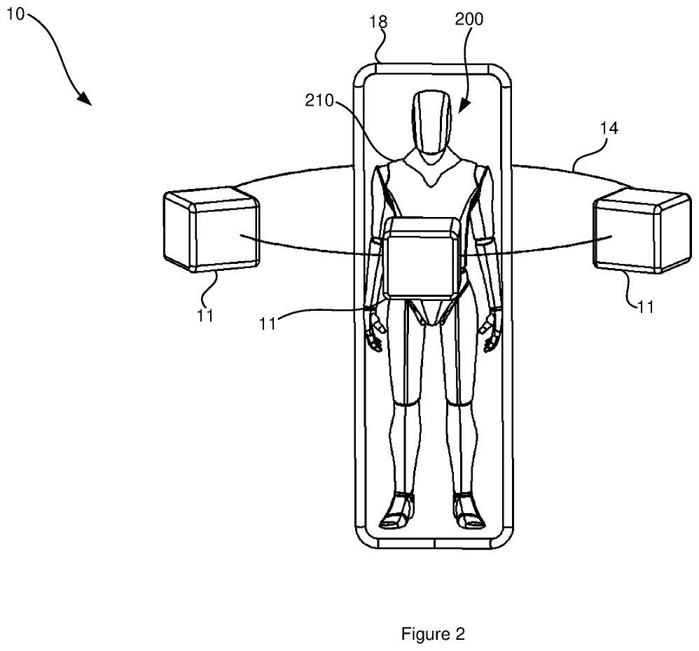

At least one pair of energy sources and detectors 110B, 120B are spatially positioned around the subject's body 210 in the second plane offset at an angle relative to the first plane having at least two pairs of energy sources and detectors 110A, 120A. Owing to at least one pair of energy sources and detectors 110B, 120B being offset in a second plane relative to the other energy sources and detectors 110A, 120A, the imaging device 100 is compact as the energy sources and detectors can be located closer together instead of within the same plane on a common arc 14 of the system 10 as shown in FIGS. 1 and 2. Although the imaging device 100 is more compact, the device 100 still acquires images suitable for use with the XV technology with multiple images being acquired from different perspectives or imaging angles through the region 230 of the subject's body 210, and that optionally reduces the use of X-rays and/or enhances scan quality. Ideally, the multiple images from different perspectives or imaging angles may be acquired simultaneously or at substantially the same time due to the spatial arrangement of the energy sources 110A, 110B and detectors 120A, 120B.

FIG. 3 is a plan view showing an imaging device including three energy sources 110A, 110B which are spatially positioned around a subject's body 210 oriented in a supine position on a tray or bed 106. The corresponding detectors have been omitted from this figure for clarity and would be located behind the tray 106 underneath the subject's body 210. The three energy sources 110A, 110B are positioned in a substantially triangular-shaped or L-shaped configuration, although other configurations are possible including irregular shapes. Two energy sources 110A are located on a common first arc 102 in a first plane through the subject's body 210. Preferably, the first plane is a transverse or horizontal plane through the subject's body 210 as shown in FIG. 3. The energy source 110B is located on a second arc 104 in a second plane of the subject's body 210. A central energy source 110A positioned above the energy source 110B is located on both of the first arc 102 and second arc 104, thus being positioned in both of the first and second planes. Preferably, the second plane is a sagittal or vertical plane through the subject's body 210 as shown in FIG. 3. A similar arrangement is provided by the corresponding detectors 120A, 120B (omitted, see e.g., FIG. 5).

In this embodiment, the controller 140 may be configured to acquire the images using three imaging angles or perspectives through the region 230 of the subject's body 210. The imaging angles may be defined by the spatial positioning of the pairs of energy sources and detectors around the subject's body 210. Two imaging angles may be provided in the first plane through the subject's body 210 by the provision of two pairs of energy sources and detectors 110A, 120A (detectors omitted) located on the first arc 102. Furthermore, one additional imaging angle may be provided in the second plane through the subject's body 210 by the provision of one pair of energy sources and detectors 110B, 120B (detectors omitted) located on the second arc 104. The imaging angles may be defined by the imaging or projection line connecting the energy source 110 and corresponding detector 120, which passes through the region 230 of the subject's body 210 to be imaged, as shown by imaging beams 116 in the embodiments of FIGS. 5 to 12 (see also e.g., imaging beams 16 of FIGS. 1 and 2).

The two imaging angles in the first plane defined by the imaging lines through the subject's body 210 connecting the two pairs of energy sources and detectors 110A, 120A may preferably be spaced apart in a range of about 45 to 90 degrees. Preferably, the two imaging angles are spaced apart in a range of about 45 to 70 degrees or about 70 to 90 degrees, or about 45 to 60 degrees, about 60 to 70 degrees, about 70 to 80 degrees or about 80 to 90 degrees. The spacing may be about 45 degrees, about 50 degrees, about 55 degrees, about 60 degrees, about 65 degrees, about 70 degrees, about 75 degrees, about 80 degrees, about 85 degrees or about 90 degrees. Preferably, the spacing is about 80 degrees. However, in other embodiments, the spacing may be preferably about 60 degrees, depending on the spatial positioning of the two pairs of energy sources and detectors in the first plane.

The two energy sources 110A and the two detectors 120A (not shown) in the first plane may be each located on a respective common arc in the first plane, which may be the same common arc, namely the first arc 102 as shown in FIG. 3. Similarly, the two energy sources 110A (central source), 110B and the two detectors 120A, 120B (not shown) in the second plane may each be located on a respective common arc in the second plane, which may be the same common arc, namely the second arc 104 as shown in FIG. 3. Thus, in this embodiment, the subject 200 may be positioned centrally within the imaging device 100 and equidistant from each of the energy sources 110A, 110B and detectors 120A, 120B.

The imaging process of FIG. 3 is more clearly demonstrated by the embodiments of FIGS. 5 to 12 which include four energy sources 110A, 110B and four detectors 120A, 120B. Each energy source 110A, 110B produces an imaging beam 116 which passes through the region 230 to be imaged and a projection is acquired by a corresponding detector 120A, 120B. Each energy source 110A, 110B is angled towards the region 230 to be imaged so that the imaging beams 116 are received through the same volume, which is the area of interest being imaged by all sources 110A, 110B, although from different angles or perspectives.

In the embodiments of FIGS. 3 to 9, the energy sources 110A, 110B are angled towards the region 230 to be imaged, and the corresponding detectors 120A, 120B are angled towards the respective energy sources 110A, 110B in order to acquire the images. Each of the detectors 120A, 120B are substantially aligned with the respective energy sources 110A, 110B, and in fact, directly face the respective energy sources 110A, 110B. The detectors 120A, 120B are substantially aligned with the respective energy sources such that the imaging beams 116 generated by the respective energy sources 110A, 110B are substantially orthogonal to the detectors 120A, 120B.

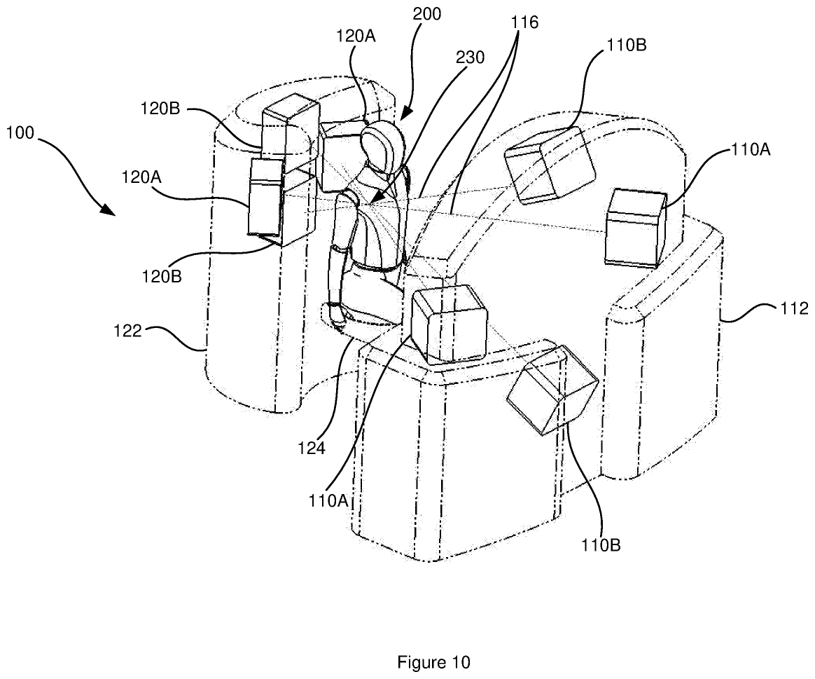

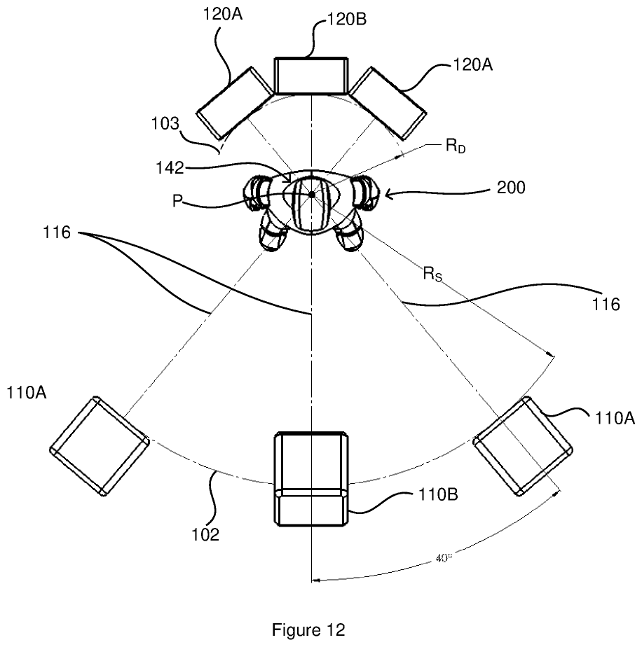

In contrast, the embodiments of FIGS. 10 to 12 show an alternative arrangement in which two detectors 120B are angled relative to the respective energy sources 110B. The detectors 120B may indirectly face the respective energy sources 110B. The detectors 120B may be angled such that the imaging beams 116 generated by the energy sources 110B are not substantially orthogonal with the detectors 120B. Furthermore, the detectors 120B are not located on a common arc in the second plane (in contrast to the detectors 120A on arc 103 as shown in FIG. 12). However, all of the energy sources 110A, 110B are located on common arc 102 or 104. Nonetheless, the two detectors 120B are spatially positioned and angled relative to the respective energy sources 110B such that they still receive the imaging beam 116 passing through the region 230 from the respective energy sources 110B.

FIG. 4 is a plan view showing another imaging device 100 including four energy sources 110A, 110B which are spatially positioned around a subject's body 210 oriented in a supine position on a tray or bed 106. The corresponding detectors have been omitted from this figure for clarity and would be located behind the tray 106 underneath the subject's body 210. The four energy sources 110A, 110B are positioned in a substantially T-shaped configuration. Three energy sources 110A are located on a common first arc 102 in a first plane through the subject's body 210. Preferably, the first plane is a transverse or horizontal plane of the subject's body 210 as shown in FIG. 4. The energy source 110B is located on a second arc 104 in a second plane of the subject's body 210. The central energy source 110A on the first arc 102 is also positioned on the second arc 104, and thus is provided in both of the first and second planes. Preferably, the second plane is a sagittal or vertical plane through the subject's body 210 as shown in FIG. 4. A similar arrangement may be provided by the corresponding detectors 120A, 120B (omitted, see e.g., FIG. 5).

In this embodiment, the controller 140 may be configured to acquire the images using four imaging angles or perspectives through the region 230 of the subject's body 210. The imaging angles may be defined by the spatial positioning of the pairs of energy sources and detectors around the subject's body 210. Three imaging angles may be provided in the first plane through the subject's body 210 by the provision of three pairs of energy sources and detectors 110A, 120A (detectors omitted) located on the first arc 102. Furthermore, one additional imaging angle may be provided in the second plane through the subject's body 210 by provision of one pair of energy sources and detectors 110B, 120B (detectors omitted) located on the second arc 104. The imaging angles may be defined by the imaging or projection line connecting the energy source 110 and detector 120, which passes through the region 230 of the subject's body 210 to be imaged, as shown by imaging lines 116 in the embodiments of FIGS. 5 to 12.

The three imaging angles in the first plane defined by the imaging lines through the subject's body 210 connecting the three pairs of energy sources and detectors 110A, 120A may preferably be each spaced apart in a range of about 45 to 90 degrees. Preferably, the three imaging angles are each spaced apart in a range of about 45 to 70 degrees or about 70 to 90 degrees, or about 45 to 60 degrees, about 60 to 70 degrees, about 70 to 80 degrees or about 80 to 90 degrees. The spacing may be about 45 degrees, about 50 degrees, about 55 degrees, about 60 degrees, about 65 degrees, about 70 degrees, about 75 degrees, about 80 degrees, about 85 degrees or about 90 degrees. Preferably, the spacing is about 80 degrees. However, in other embodiments, the spacing may be preferably about 60 degrees, depending on the spatial positioning of the three pairs of energy sources and detectors in the first plane.

The three energy sources 110A and the three detectors 120A (not shown) in the first plane may be each located on a respective common arc in the first plane, which may be the same common arc, namely the first arc 102 as shown in FIG. 4. Similarly, the two energy sources 110A (central source), 110B and the two detectors 120A, 120B (not shown) in the second plane may each be located on a respective common arc in the second plane, which may be the same common arc, namely the second arc 104 as shown in FIG. 4. Thus, in this embodiment and similar to FIG. 3, the subject 200 may be positioned centrally within the imaging device 100 and equidistant from each of the energy sources 110A, 110B and detectors 120A, 120B. The detectors 120A, 120B are substantially aligned with the respective energy sources 110A, 110B in this embodiment and are positioned orthogonally to the imaging beams 116 generated by the respective energy sources 110A, 110B. However, the detectors 120A, 120B may not be substantially aligned and instead angled relative to the respective energy sources 110A, 110B as will be described in relation to FIGS. 10 to 12.

In the embodiments of FIGS. 3 and 4, the second plane is orthogonal to the first plane such that the first and second arcs 102 and 104 are at 90 degrees relative to one another and the single energy source 110B is aligned below the central energy source 110A on the second arc 104. However, in other embodiments, the second plane may be offset at an angle in a range of between about 70 to 90 degrees relative to the first plane. Preferably, the offset angle is about 80 degrees. Thus, the energy source 110B may be angled relative to the central energy source 110A by an angle of about 20 degrees to the left or right of a vertical or sagittal plane through the subject's body, or preferably, about 10 degrees to the left or right of the vertical or sagittal plane. The three energy sources 110A, 110B (and three detectors 120A, 120B not shown) of FIG. 3 may not form an exact L-shaped configuration, and instead may form a substantially L-shaped configuration due to angling of the energy source 110B relative to the central energy source 110A. Similarly, the four energy sources 110A, 110B (and four detectors 120A, 120B not shown) of FIG. 4 may not form an exact T-shaped configuration as the vertical line of the ‘T’ may be angled relative to the horizontal line of the ‘T’, and instead may form a substantially T-shaped configuration due to angling of the energy source 110B relative to the central energy source 110A.

In other embodiments, the energy source 110B may be aligned above the central energy source 110A on the second arc 104 (not shown) in the embodiments of FIGS. 3 and 4. In relation to FIG. 4, the energy sources 110A, 110B and detectors (not shown) may form an inverted T-shaped configuration. The energy source 110B of FIGS. 3 and 4 may be angled relative to the central energy source 110A by an angle of about 20 degrees to the left or right of a vertical or sagittal plane through the subject's body, or preferably, about 10 degrees to the left or right of the vertical or sagittal plane. Thus, the four energy sources 110 may not form an exact inverted T-shaped configuration due to the angling of the energy source 110B. By varying the angles of the individual sources 110A, 110B and detectors 120A, 120B, various shaped configurations may be produced, including irregular or asymmetric shapes as will be described below.

Although not shown in FIG. 3, three corresponding detectors would also be provided in the imaging device 100, where the three detectors form an approximately triangular-shaped or L-shaped configuration. Similarly, although not shown in FIG. 4, four corresponding detectors would also be provided in the imaging device 100, where the four detectors may also form an approximately T-shaped or inverted T-shaped configuration.

Although FIGS. 3 and 4 depict offset angles of the second plane relative to the first plane angles of about 90 degrees (and preferably between about 70 to about 90 degrees), these are not limiting. The second plane may be offset at an angle of about 70 degrees, about 75 degrees, about 80 degrees, about 85 degrees or about 90 degrees. The second plane may be offset at an angle of about 70 to 80 degrees or of about 80 to 90 degrees relative to the first plane. The second plane may be offset at an angle of about 80 degrees relative to the first plane.

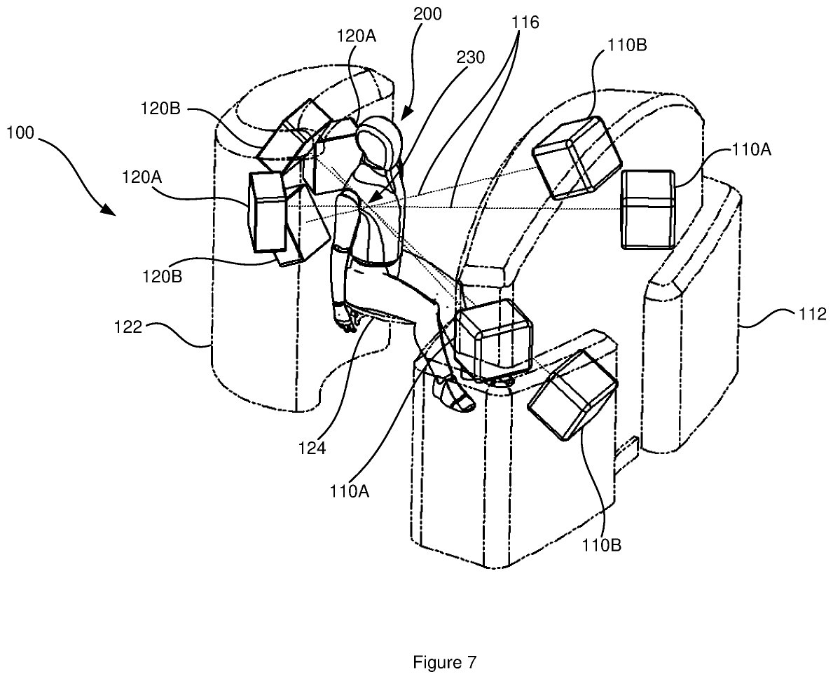

The energy sources 110A on the first arc 102 may also be spaced further apart up to 180 degrees circumferentially around the subject's body 210. In an alternative arrangement, the energy sources 110A may be spaced apart beyond 180 degrees such that one energy source 110A is located behind the subject's body 210 and a corresponding detector 120A is located in front of the subject's body 200. However, it is preferable that the energy sources 110A, 110B are closely positioned in order to provide a more compact scanner 100. Furthermore, the configuration of the energy sources 110A, 110B is also reflected in the corresponding arrangement of the detectors 120 (not shown). Thus, the detectors 120 are also ideally closely positioned in order to provide a more compact scanner 100. This will be explained in more detail in relation to an exemplary source unit 112 and detector unit 122 as shown and described with respect to FIGS. 7, 10 and 13.

FIG. 5 is a perspective view of another imaging device 100 showing four energy sources 110 (denoted as 110A, 110B) and four detectors 120 (denoted as 120A, 120B) each spatially positioned around a subject's body 210 in a diamond-shaped configuration, where the subject's body 210 is oriented in an upright standing position in the scanner 100. The imaging device 100 includes two pairs of energy sources and detectors 110A, 120A and two pairs of energy sources and detectors 110B, 120B. The two pairs of energy sources and detectors 110A, 120A are spatially positioned in a first plane around the subject's body 210. The first plane is preferably a transverse or horizontal plane through the subject's body 210 as shown in FIG. 5. The two pairs of energy sources and detectors 110B, 120B are spatially positioned in a second plane around the subject's body 210. The second plane is preferably a sagittal or vertical plane through the subject's body 210. As shown in FIG. 5, the first and second planes intersect through the region 230 of the subject's body 210 to be imaged, as indicated by the intersection of imaging beams 116 between the respective energy source and detector pairs.

In this embodiment, the controller 140 may be configured to acquire the images using four imaging angles or perspectives through the region 230 of the subject's body 210. Two imaging angles may be provided in the first plane through the subject's body 210 by the provision of two pairs of energy sources and detectors 110A, 120A. Furthermore, two imaging angles may be provided in the second plane through the subject's body 210 by the provision of two pairs of energy sources and detectors 110B, 120B. The imaging angles may be defined by the imaging or projection lines connecting the energy sources 110 and detectors 120, which pass through the region 230 of the subject's body 210 to be imaged, as indicated by the imaging beams 116.

In the embodiments shown in FIGS. 5 to 12 which include four energy sources and four detectors, the energy sources 110A, 110B and detectors 120A, 120B are not provided on the same common arcs 102, 104 in the first and second planes in contrast to the embodiments of FIGS. 3 and 4. This is because the imaging devices 100 of FIGS. 3 and 4 enable the subject 200 to be centrally located between the energy sources 110 and detectors 120, whereas the imaging devices 100 of FIGS. 5 to 12 are configured to accommodate the subject 200 between the energy sources 110 and detectors 120 in a position that is closer to the detectors 120 than the energy sources 110.

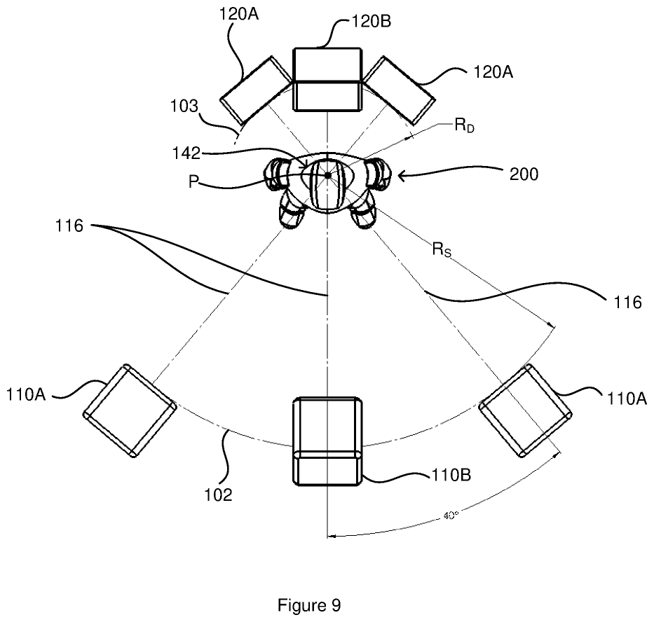

As shown in FIGS. 5 to 12, the pair of energy sources 110A may be provided on the first arc 102 and the pair of energy sources 110B may be provided on the second arc 104. However, the corresponding detectors pairs 120A, 120B may be provided on different common arcs from those of the first and second arcs 102, 104. This is best observed in the embodiments of FIG. 9 showing a plan view of the arrangement of the imaging device 100 of FIG. 7. The detector pairs 120A may be provided on a common arc 103 and the detector pairs 120B may be provided on another common arc (not shown). Where the energy sources 110A, 110B and detectors 120A, 120B are located on different common arcs, the length of the common arcs 102, 104 on which the energy sources are located preferably have a greater length than the common arcs (see arc 103 and other common arc not shown) on which the detectors are located. Thus, in the embodiments of FIGS. 5 to 12, the subject 200 may be located in closer proximity to the detectors 120A, 120B than the energy sources 110A, 110B within the imaging device 100. This will be described in more detail in relation to FIGS. 9 and 12.

Notably, the energy sources and detectors need not be provided on common arcs 102, 104 in the first and second planes and optionally, may not be aligned in the first and second planes around the subject's body 210, as would be appreciated by a person skilled in the art.

In the embodiment of FIG. 5, the two pairs of energy sources and detectors 110A, 120A in the first plane provide imaging angles that are circumferentially spaced apart at an angle of about 80 degrees. Furthermore, the two pairs of energy sources and detectors 110B, 120B in the second plane provide imaging angles that are circumferentially spaced apart at an angle of about 60 degrees as indicated.

FIG. 5 shows a diamond-shaped configuration of the energy sources 110A, 110B and the detectors 120A, 120B where the diamond is in the form of an addition or ‘plus’ sign centred relative to the region 230 of the subject's body 210 to be imaged at the intersection of the first and second planes. The imaging beams 116 generated by the energy sources 110A, 110B intersect through an intersection region 142, which may include a single intersection point P (see also FIGS. 9 and 12). The intersection region 142 of the imaging device 100 will correspond to the region 230 of the subject's body 210 to be imaged. The location of the intersection region 142 and intersection point P is dependent on the spatial arrangement of the energy sources 110A, 110B and detectors 120A, 120B, which can be selected based on a desired positioning of the subject 200 in the imaging device 100, as will be described in relation to FIGS. 9 and 12.

The first plane may be a horizontal or transverse plane and the second plane may be in a vertical or sagittal plane of the subject's body 210 as located in an upright standing position as shown in FIG. 5. The energy sources 110A may be circumferentially spaced about 40 degrees to the left or right of the intersection of the first arc 102 with the second arc 104. Furthermore, the energy sources 110B may be circumferentially spaced about 30 degrees above or below of the intersection of the second arc 104 with the first arc 102. Similar circumferential spacing may be provided with respect to the detectors 120A, 120B on their respective common arcs in the first and second planes (see e.g., common arc 103 for detectors 120A in FIGS. 9 and 12).

Although FIG. 5 depicts angles of about 60 and 80 degrees between the imaging angles or perspectives provided by the pairs of energy sources and detectors, embodiments of the disclosure are not limited to these angles, or to providing circumferential spacing on an arc in the planes. The imaging angles may be spaced further apart up to 180 degrees circumferentially around the subject's body 210. However, it is preferable that the energy sources 110A, 110B are closely positioned in order to provide a more compact scanner 100. Furthermore, the configuration of the energy sources 110A, 110B is also reflected in the corresponding arrangement of the detectors 120A, 120B as shown by the imaging beams 116 through the region 230. Thus, the detectors 120A, 120B are ideally closely positioned in order to provide a more compact scanner 100. This will be explained in more detail in relation to an exemplary source unit 112 and detector unit 122 as shown and described with respect to FIGS. 7, 10 and 13.

In some embodiments, the imaging angles provided by the pairs of energy sources and detectors 110A, 120A in the first plane may be spaced apart in a range of about 45 to 90 degrees, being preferably around 80 degrees apart in the diamond-shaped configuration as shown in FIG. 5. Although not shown, various other configurations of the energy sources and detectors may be provided such as a rectangular-shaped configuration, or an oval or elliptical-shaped configuration where additional energy sources and detectors are provided. Furthermore, irregular-shaped configurations may be provided.

In the diamond-shaped configuration of FIG. 5, the two imaging angles provided by the pairs of energy sources and detectors 110A, 120A may be spaced apart in the first plane in a range of about 45 to 70 degrees or about 70 to 90 degrees, or about 45 to 60 degrees, about 60 to 70 degrees, about 70 to 80 degrees or about 80 to 90 degrees. The spacing may be about 45 degrees, about 50 degrees, about 55 degrees, about 60 degrees, about 65 degrees, about 70 degrees, about 75 degrees, about 80 degrees, about 85 degrees or about 90 degrees. However, preferably the spacing is about 80 degrees as shown in FIG. 5 for the diamond-shaped configuration.

Furthermore, the two imaging angles provided by the pairs of energy sources and detectors 110B, 120B may be spaced apart in the second plane in a range of about 45 to 70 degrees. Preferably, the spacing is in a range of about 45 to 60 degrees or about 60 to 70 degrees. The spacing may be about 45 degrees, about 50 degrees, about 55 degrees, about 60 degrees, about 65 degrees or about 70 degrees. Preferably, the spacing is about 60 degrees as shown in FIG. 5 for the diamond-shaped configuration.

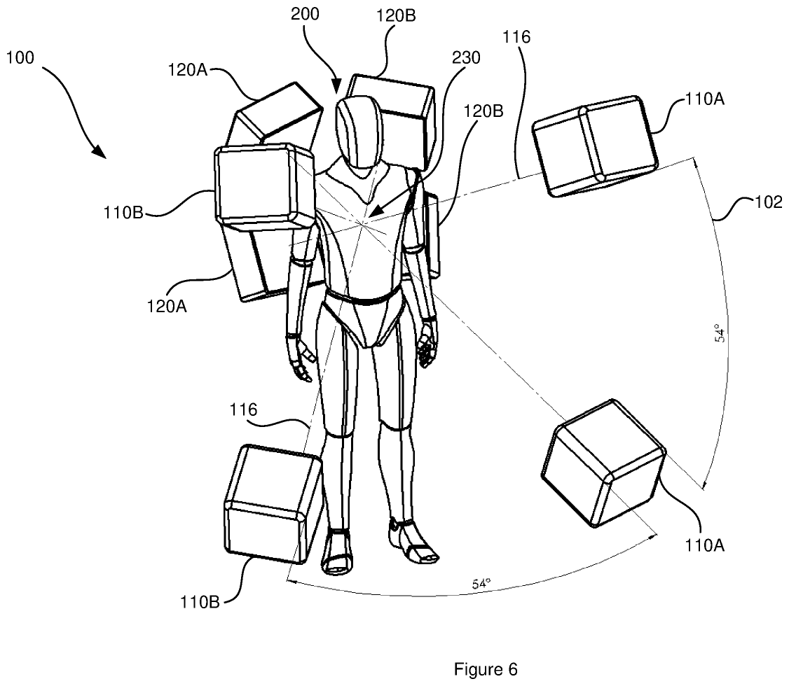

FIG. 6 is a perspective view of another imaging device 100 showing four energy sources 110 (denoted as 110A, 110B) and four detectors 120 (denoted as 120A, 120B) each spatially positioned around a subject's body 210 in a square-shaped configuration, where the subject's body 210 is oriented in an upright standing position in the scanner 100. Two pairs of energy sources and detectors 110A, 120A are spatially positioned around the subject's body 210 in a first plane and two pairs of energy sources and detectors 110B, 120B are spatially positioned around the subject's body 210 in a second plane. The first and second planes are angled relative to a sagittal or vertical plane of the subject's body 210. The second plane is offset at an angle of 54 degrees relative to the first plane, as indicated between the spacing of energy sources 110A and 110B near the subject's feet.

In relation to the square-shaped configuration of FIG. 6, four imaging angles may be provided by the pairs of energy sources and detectors 110A, 110B, and 120A, 120B which are spaced apart in the first and second planes in a range of about 45 to 70 degrees, being preferably around 54 degrees as shown. Preferably, the four imaging angles are spaced apart in a range of about 45 to 60 degrees or about 60 to 70 degrees. The spacing may be about 45 degrees, about 50 degrees, about 55 degrees, about 60 degrees, about 65 degrees or about 70 degrees. Preferably, the spacing is about 60 degrees, and more preferably about 54 degrees as shown in FIG. 6 in the square-shaped configuration.

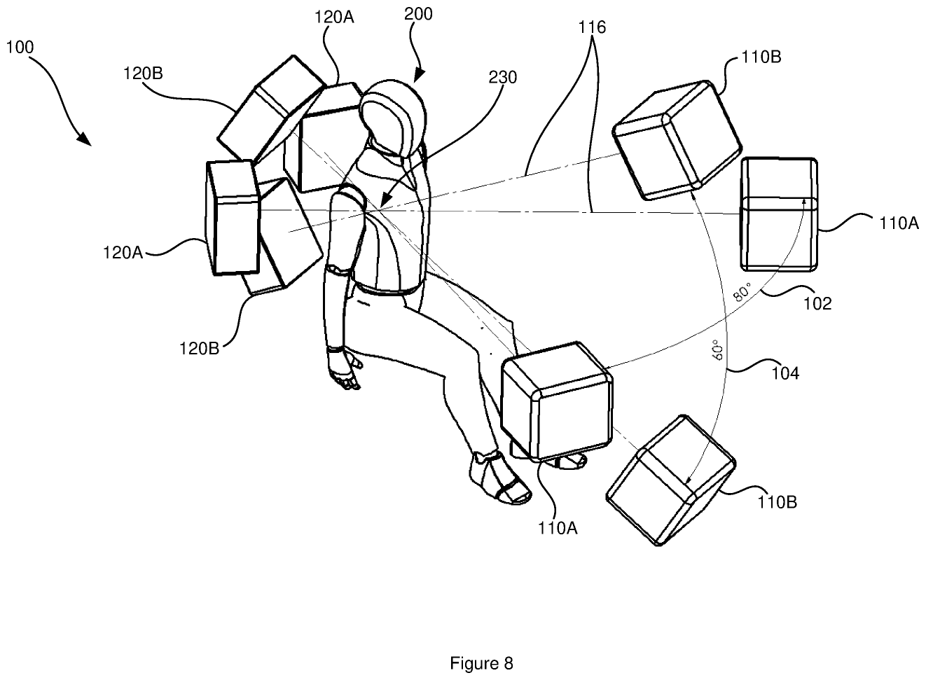

Turning to FIGS. 7 to 9, another imaging device 100 has four energy sources 110 (denoted as 110A, 110B) positioned in an exemplary source unit 112 and four detectors 120 (denoted as 120A, 120B) positioned in an exemplary detector unit 122 of the imaging device 100. The source unit 112 and detector unit 122 are shown in broken lines indicating that this is only an exemplary embodiment of the shape and location of these units in the scanner 100. The four energy sources 110 and four detectors 120 are each spatially positioned around the subject's body 210 in a diamond-shaped configuration as described in relation to the embodiment of FIG. 5. However, the subject's body 210 is now oriented in an upright seated position in the scanner 100 which includes a seat or chair 124 as part of the detector unit 122. FIG. 8 shows the same imaging device 100 of FIG. 7 although excludes the source unit 112 and detector unit 122 for clarity. The imaging angles and/or angles between the energy sources 110 and/or detectors 120 may be substantially similar to those of the diamond-shaped configuration described in relation to the embodiment of FIG. 5.

As can be observed in FIG. 7, the imaging device 100 is configured to accommodate the subject 200 in an upright orientation between the energy sources 110 and detectors 120. The subject 200 may be positioned on a seat 124 of the detector unit 122 for image acquisition. In alternative embodiments, the seat 124 may be excluded and able-bodied subjects 200 may be able to walk into the scanner 100 and position themselves in a standing position between the source unit 112 and detector unit 122 for image acquisition. In some embodiments, the energy sources 110 are spaced approximately 1200 mm relative to the patient's spine, while the detectors 120 are spaced approximately 400 mm relative to the patient's spine. This provides a sufficient gap of at least 1000 mm between the source unit 112 and detector unit 122 for the subject 200 to walk into and/or be positioned in the scanner 100.

FIG. 9 shows the imaging device 100 of FIG. 7 in a plan view excluding the source unit 112 and detector unit 122 for clarity. FIG. 9 illustrates that the imaging beams 116 generated by the energy sources 110A, 110B intersect through an intersection region 142, which may include a single intersection point P. The intersection region 142 of the imaging device 100 will correspond to the region 230 of the subject's body 210 to be imaged. The intersection point P is not equidistant from each of the energy sources 110A, 110B and detectors 120A, 120B. In the embodiments of FIGS. 5 to 12, the intersection point P is located closer to the detectors 120A, 120B than the energy sources 110A, 110B (in comparison to FIGS. 3 and 4 in which the intersection point P would be equidistant from the energy sources and detectors). A radius of curvature from the intersection point P to the common arc 103 on which the pair of detectors 120A are located, denoted as RD, may be about 400 mm, or more particularly, about 410 mm. A radius of curvature from the intersection point P to the first arc 102 on which the pair of sources 110A are located, denoted as RS, may be about 1200 mm.

The advantage of having the intersection region 142 and more particularly, the intersection point P, being closer to the detectors 120A, 120B than the energy sources 110A, 110B, is that this reduces the magnification of the images acquired by the imaging device 100. Magnification occurs when the energy sources 110A, 110B are positioned too close to the region being imaged, e.g., the region 230 of the subject 200, and the image captured exaggerates the size and dimensions of the structures. It may be desirable to reduce the magnification in order to provide a more accurate representation of the region 230 to be imaged. A posterior-anterior (PA) projection beam view allows a more accurate representation of the region 230 to be imaged, such as particularly the heart or lungs of the subject 200, as the region 230 is positioned in closer proximity to the detectors 120A, 120B and is therefore less magnified. A person skilled in the art would appreciate that the radii of curvature Rs and RD may be varied as appropriate for the dimensions of the imaging device 100, although it remains preferable that the radius Rs is greater than the radius RD.

FIGS. 10 to 12 show another imaging device 100 having a similar arrangement to FIGS. 7 to 9, except that the two detectors 120B are angled relative to the respective energy sources 110B. The two detectors 120B indirectly face the respective energy sources 110B and are angled such that the imaging beams 116 generated by the energy sources 110B are not substantially orthogonal with the detectors 120B. While the two detectors 120B are angled towards the respective energy sources 110B, similar to the detectors 120A and respective energy sources 110A, the two detectors 120B are co-planar and vertically oriented relative to one another. More specifically, the two detectors 120B are positioned one above the other in the imaging device 100.

The detectors 120B are not provided on a common arc in a plane through the subject's body 210. In contrast, the energy sources 110A are provided on a first arc 102 in a first plane through the subject's body 210, the energy sources 110B are provided on a second arc 104 in a second plane through the subject's body 210, and the detectors 120A are provided on a different arc 103 in the second plane through the subject's body 210 as shown in FIG. 12. While the two pairs of energy sources and detectors 110A, 120A are provided in the first plane and two pairs of energy sources and detectors 110B, 120B are provided in the second plane, the detectors 120B are not provided on a common arc in the second plane.

The advantage of the alternative arrangement of FIGS. 10 to 12 is that the two co-planar detectors 120B enable the imaging device 100 to be more compact. The detector unit 122 can thus be narrower as the vertically-oriented detectors 120B, which are not located on a common arc, have less width than in the arrangement of FIGS. 7 to 9. Thus, an even smaller, more compact imaging device may be provided by this inventive embodiment that still allows for multiple images to be acquired at different angles through the subject's body 210. In other embodiments (not shown), the two detectors 120A may be angled relative to the respective energy sources 110A. The two detectors 120A may indirectly face the respective energy sources 110A and be angled such that the imaging beams 116 generated by the energy sources 110A are not substantially orthogonal with the detectors 120A. The two detectors 120A may be co-planar and horizontally oriented relative to one another. In some embodiments (not shown), all of the detectors 120A, 120B may be co-planar relative to one another while remaining angled towards the respective energy sources 110A, 110B to acquire the images. This may advantageously further reduce the width of the detector unit 122, thereby providing a more compact and smaller imaging device 100.

In relation to dynamic in vivo imaging of the lungs, the images of the most value include those where the individual lungs are separated on the images and there is minimal bone obstruction. Thus, the most valuable angle to image is in the sagittal or vertical plane through the subject's body as the lungs are separated by the spinal column. As the imaging angle increases relative to the spinal axis of the patient, the lungs start to overlap from about 40 degrees and with further angle increase, the spine and arms of the patient may also be included in the image. Thus, there is a necessary balance of having sufficient views or perspectives of images at suitable separation in order to reconstruct those images to show dynamic lung function. The energy sources and detectors in the scanner can be positioned closely together, by providing at least one energy source and at least one detector on a different plane to the remaining energy sources and detectors. This enables sufficient perspectives of images to be acquired for dynamic in vivo imaging, while advantageously reducing the space required.

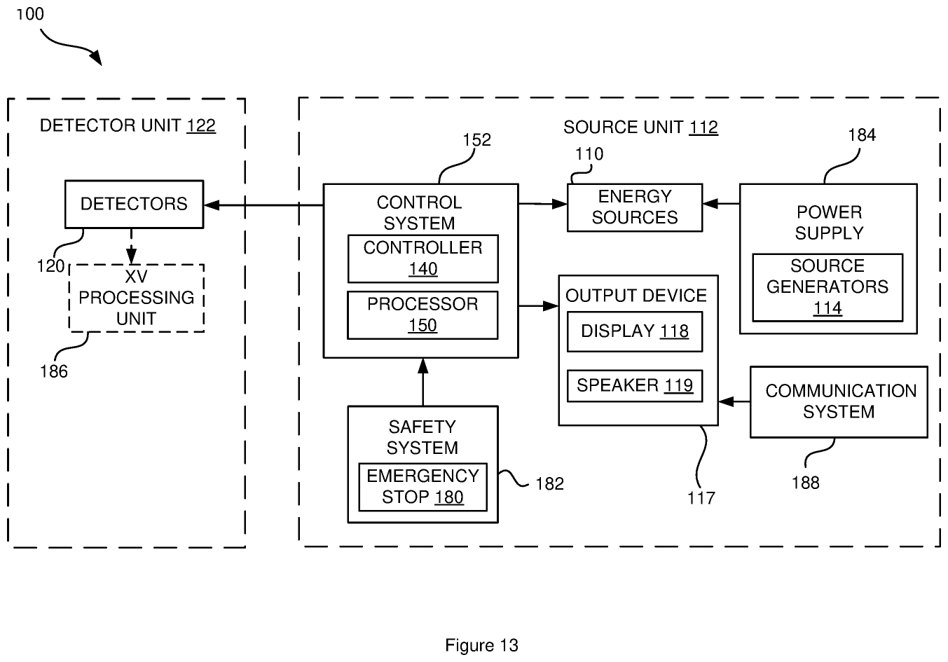

FIG. 13 is a schematic diagram of components of a source unit 112 and detector unit 122 of the imaging device 100 according to various embodiments of the disclosure which will be described in detail below.

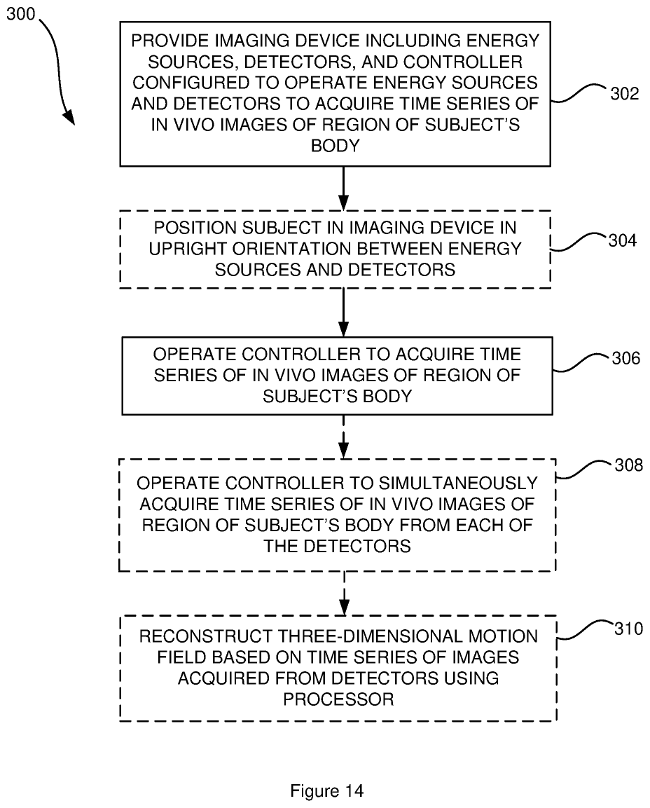

FIG. 14 illustrates a method 300 for acquiring a time series of in vivo images of a region 230 of a subject's body 210. The method 300 includes a step 302 of providing an imaging device 100 including at least three energy sources 110 (denoted as 110A, 110B) and at least three detectors 120 (denoted as 120A, 120B) for detecting energy from the at least three energy sources 110 passing through the region 230 of the subject's body 210 located between the energy sources 110 and the detectors 120. The imaging device 100 also includes a controller 140 configured to operate the at least three energy sources 110 and the at least three detectors 120 to acquire a time series of in vivo images of the region 230 of the subject's body 210. The method also includes a step 306 of operating the controller 140 to acquire the time series of in vivo images of the region 230 of the subject's body 210.

The imaging device 100 may include one or more features as described herein and in relation to the embodiments of FIGS. 3 to 13. The imaging device 100 includes at least two pairs of energy sources and detectors 110A, 120A spatially positioned around the subject's body 210 in a first plane, and at least one pair of energy sources and detectors 110B, 120B spatially positioned around the subject's body in a second plane. The first plane and the second plane intersect through the region 230 of the subject's body to be imaged.

As shown in FIG. 14, the method 300 optionally includes the step 304, performed before operating the controller 140 to acquire the images, of positioning the subject 200 in the imaging device 100 in an upright orientation between the energy sources 110 and detectors 120. For example, the subject 200 may be positioned in an upright standing position as shown in the embodiments of the imaging device 100 of FIGS. 5 and 6. Alternatively, the subject 200 may be positioned in an upright seated position in the imaging device 100 as shown in the embodiments of FIGS. 7 to 12. For able-bodied patients 200, they may simply walk into the space between the energy sources 110 and detectors 120 and sit down on the seat 124 or alternatively, position themselves in a standing or upright position for the image acquisition. For wheelchair or limited mobility patients, an operator may assist with transfer to the seat 124 or a wheelchair with radiolucent seat back may be provided and positioned in the scanner 100. After this step is complete, either the operator or the communication system 188 may advise the subject 200 of the estimated duration of the scan.

In some embodiments, the method 300 may also include two optional steps 308 and 310 as shown in broken lines in FIG. 14. The method 300 may include the step 308 of operating the controller 140 to acquire a time series of in vivo images of the region 230 of the subject's body 210 simultaneously or at substantially the same time from each of the detectors 120. The controller 140 is configured to acquire at least three time series of in vivo images of the region 230 of the subject's body 210. However, in some embodiments where the imaging device 100 includes four energy sources 110 and four detectors 120, the controller is configured to acquire four time series of in vivo images of the region 230 of the subject's body 210.

Multiple time series of images may be advantageously acquired by the imaging device 100 and method 300 simultaneously or at substantially the same time over part of the breath or over a full breath of the subject 200. Preferably, the time series of images are acquired over a full single breath of the subject 200. Acquiring multiple time series (from different angles) of a single breath, rather than acquiring a single time series (from different angles) of multiple breaths, removes the requirement for the subject 200 to maintain consistent breathing across multiple breaths. The controller 140 may operate each energy source 110 and corresponding detector 120 to acquire the images at the same or substantially the same time. Instead of operating the energy sources 110 and corresponding detectors 120 simultaneously, it may be preferable to sequentially acquire the images with a short timing offset for operation of the energy source/detector pairs. This may advantageously reduce x-ray backscatter and thus improve the image quality. The processor 140 may be configured to correct for the timing differences between the time series of images acquired when processing the data. Advantageously, for imaging devices 100 employing the use of x-rays, this reduces the radiation dosage as all of the energy sources 110 and corresponding detectors 120 may be simultaneously or at substantially the same time operated by the controller 140 for a short time to acquire the images.

Once the scan has finished after step 308, the image data may be uploaded to the XV processing unit 186, which is located either on-board the imaging device 100 or accessed via a cloud-based server and XV processing application. This step 310 may be initiated upon action taken by the operator or the processor 150 may be configured to automatically upload the image data once the scanning is complete. As shown in FIG. 14, the method 300 may also include the step 310 of using a processor 150, 186 (see FIG. 13) or off-board XV processing application to reconstruct a three-dimensional motion field of the region 230 of the subject's body 210 based on the time series of images acquired from the detectors 120 in step 308. This may employ XV processing techniques described in previously mentioned International Patent Publication Nos. WO 2011/032210 A1 and WO 2015/157799 A1 and incorporated herein by reference. The processor 150 may produce three-dimensional (i.e., three spatial dimensions) motion measurements (e.g., displacement or velocity measurements) over the time of the region 230 that was imaged (which would result in four-dimensional measurements, i.e., three spatial dimensions plus time). In addition, the three-dimensional motion measurements may have either one component of velocity (3D1C), two components of velocity (3D2C), or preferably three components of velocity (3D3C).

### Obtaining Images for Geometric Structure Image

Arrangements of source-detector pairs for the acquisition of a time series of in vivo images according to various examples have been described in relation to FIGS. 3 to 12. Although these examples provide at least 3 energy sources and 3 energy detectors, in some embodiments 2 energy sources and 2 energy detectors may suffice for the acquisition of the time series of images. As discussed, this time series of images can be used to reconstruct a motion measurement, such as a three-dimensional motion field of the region 230 of the subject's body which has utility in functional imaging for evaluation of e.g. the lungs. However, this type of motion measurement can be of limited value when considered without context such as the location and orientation of the lung. Thus, the present disclosure relates to an improvement in imaging devices and methods that enables acquisition of a second set of images for construction of a geometric structure image. It is preferred that at least one of the source-detector pairs is used to acquire images for both the time series of in vivo images and the second series of images for construction of the geometric structure image. The geometric structure image can be combined with the motion measurement or motion field representations to provide a hybrid visualisation that presents not only a functional view of the region of interest, but also presents a geometrical structural view of the region, ideally a three-dimensional geometrical structural view.

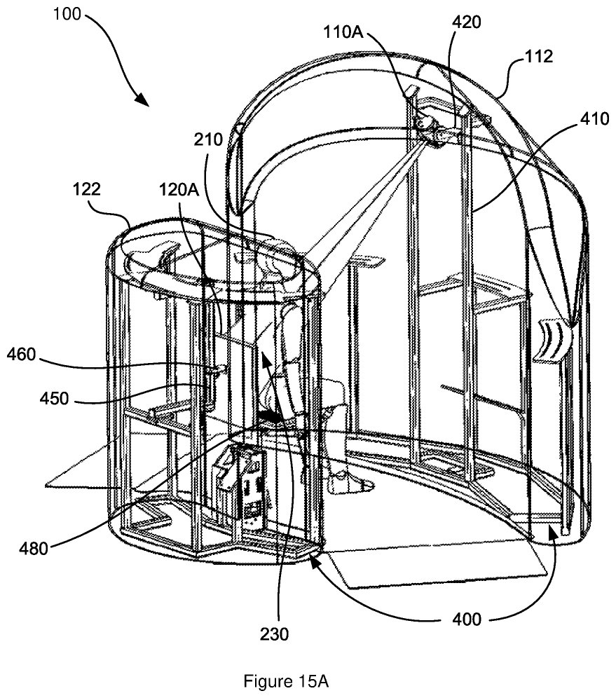

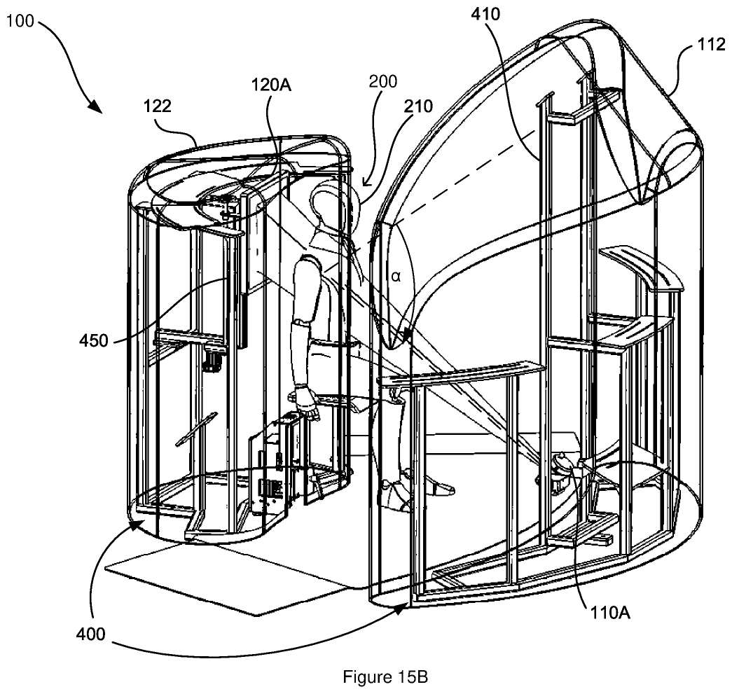

Thus, in one embodiment for acquisition of the time series of in vivo images the imaging device 100 comprises at least two energy sources and at least 2 energy detectors for detecting energy from the at least two energy sources passing through the region of the subject's body located between the energy sources and the energy detectors. The first source-detector pair is located in a first plane, and the second source-detector pair is located in a second plane, wherein the first plane and the second plane intersect through the region 230 of the subject's body 210 to be imaged. Additionally, for acquisition of the second set of images for construction of a geometric structure image, a motion assembly 400 is provided, which is configured to achieve relative movement between at least one of the source-detector pairs and the subject 200 as will be described with reference to FIGS. 15A and 15B. Thus, as noted previously, at least one of the source-detector pairs is used to acquire images for both the time series of in vivo images and the second series of images for construction of the geometric structure image.

Since use of the source-detector pairs for obtaining the time series of images has already been explained, for simplicity the imaging device 100 of FIGS. 15A and 15B which incorporates a motion assembly 400 has omitted all but one of the source-detector pairs used to obtain the time series of images. The omitted source-detector pairs remain stationary for the purpose of obtaining the time sequence of images as previously described. However, in order to obtain the second set of images required for construction of the geometric structure image, a motion assembly 400 is provided to achieve relative movement between at least one source-detector pair and the subject 200. Operation of the motion assembly 400 in coordination with operation of the source-detector pair enables the imaging device 100 to obtain images at each of a plurality of image angles in the plane of the source-detector pair. The motion assembly 400 and the energy source(s) 110A and detector(s) 120A are under control of a controller (140 in FIG. 13) in order to coordinate relative movement by the motion assembly 400 with the operation of the source 110A and detector 120A to obtain the second set of images at the plurality of angles.

Ideally, the time series of in vivo images are used to generate a motion measurement of the region 230 as described previously and the second set of images obtained while operating the motion assembly 400 are used to construct a geometric structure image of the region 230. Preferably, this is a 3D geometric structure image which, when applied over or in conjunction with the motion measurement (which may be represented in a visible motion field), gives context and richer clinical detail to the motion measurement by conveying the geometrical boundaries of the region of interest 230, as well as its position relative to the motion field. Thus, in some embodiments where the region of interest 230 is the lungs, the time series of images may be used to produce a motion measurement that visually presents regional expansion measurements determined from the time series of images, and this can be combined (e.g. overlaid) with the geometric structure image (e.g. represented as a transparent, semi-transparent or outline image) determined from the second series of images in order to provide a clinician with a visual representation of how each region of the lung is expanding.

The motion assembly 400 may comprise one or more frames for supporting one or more sources 110 and/or detectors 120, with the frames providing a track for movement of the source 110 and/or detector 120 relative to the subject 200, for acquisition of the second set of images. In some embodiments, a source frame 410 supports a source 110A by use of a coupling 420 and provides a track for vertical movement of the source 110A to emit energy for acquisition of the images at the plurality of angles required for second set of images. The controller 140 controls operation of a linear actuator on the source frame 410, or a mechanism that transfers power such as a gear, chain or pulley configured to translate the source 110A up and down the source frame 410. In some embodiments, a hydraulic or gravity system with positional feedback may be used, as would be understood by one of skill in the art. FIGS. 15A,B show a straight frame which may be desirable where it is necessary to conserve space. However, in order for the source 100A to deliver energy through the region of interest as it translates along the frame, the source emission angle must be adjusted. Accordingly, the source 110A is ideally coupled to the source frame 410 or to the linear actuator on the frame with an angular or rotary actuator that is also under control of the controller 140 and enables adjustment of the angular orientation (rotational adjustment) of source 110A to direct emitted energy through the iso-centre of the region of interest 230 as the source 110A translates along the source frame 410. In other embodiments, the source frame 410 may comprise an arc, as will be described below, in which case rotational adjustment control of the source 110A may be omitted in some embodiments.

As will be apparent from the views in FIGS. 15A and 15B, as the source 110A translates along the source frame 410, it loses alignment with detector 120A. Therefore, the motion assembly 400 may also comprise a detector frame 450 that supports detector 120A by use of a coupling 460 and provides a track for vertical movement of the detector 120A to detect energy from source 110A for acquisition of the images at the plurality of angles required for the second set of images. The controller 140 controls operation of a linear actuator such as a motor or gear, chain or pulley system as described above, configured to translate the detector 120A up and down the detector frame 450. Ideally, the controller 140 controls operation of the linear actuator on source frame 410 in synchrony with the linear actuator on detector frame 450 so that the source 110A moves downwards in synchrony with the detector 120A moving upwards in order for the images acquired to remain on the iso-centre of the region 230.

Like source frame 410, detector frame 450 is straight. Therefore an angular or rotary actuator may be provided in or with the coupling 460 of detector 120A to the detector frame 450 or linear actuator although in embodiments in which the source 110A has an angular or rotary actuator configured to direct source energy at the detector 120A, rotational adjustment of the detector may not be required. In embodiments where an angular or rotary actuator is provided with the detector 120A, the angular or rotary actuator is also under control of the controller and enables the detector 120A to detect emitted energy passing through the iso-centre of the region of interest 230 as the source 110A translates along the source frame 410 and the detector 120A translates along the detector frame 450. Notably, the source 110A may travel a longer distance along source frame 410 than detector 120A may travel along detector frame 450. This is possible due to proximity to the detector 120A to the subject 200 and conveniently permits the detector frame 450 to fit into a more compact form factor.

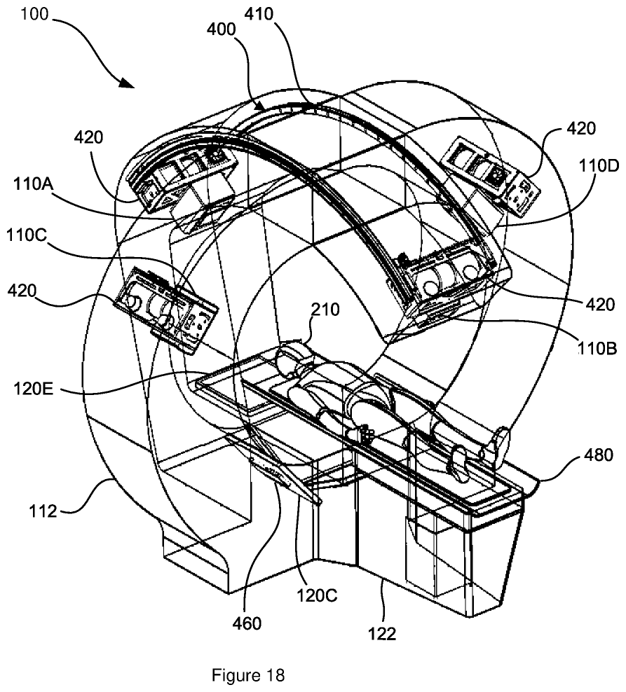

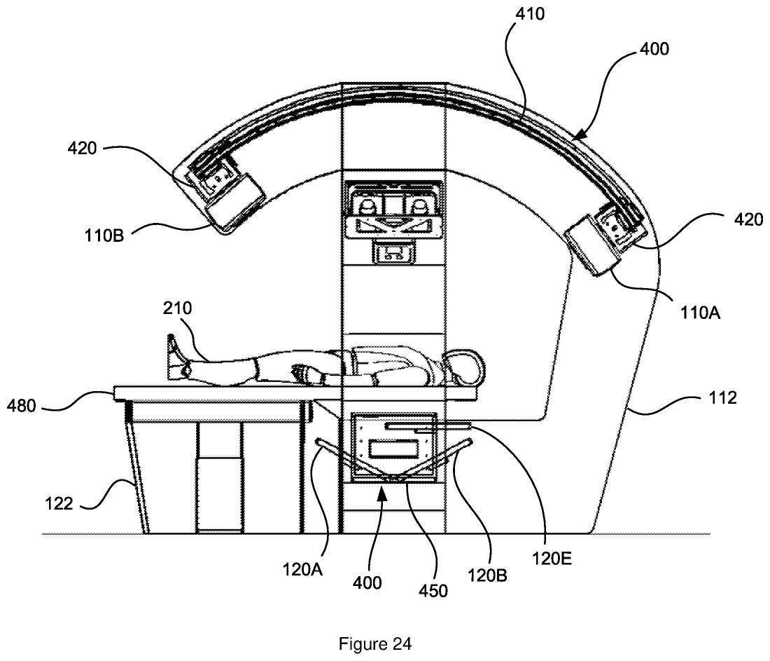

While the source frame 410 and detector frame 450 shown in FIGS. 15A and 15B are oriented vertically to obtain a plurality of images in an arc that tracks vertically (i.e. cranio-caudally of the seated subject 200), it is to be understood that the frames could be oriented differently, such as horizontally, to obtain a plurality of images in an arc that tracks horizontally (i.e. transverse of the seated subject 200). An example of such an arrangement will be described with reference to the embodiments of the imaging device in FIGS. 18 to 24. Horizontal movement of the sensor-detector pair may be preferable e.g. when obtaining a set of images for construction of a geometric structure image of the lung, due to the chest symmetry. Horizontal movement may also be preferred due to the reduced risk of exposing radiation to the subject's head, thyroid, gonads and other organs. However, the length of the sensor frame 410 may make the footprint of the imaging device 100 relatively large which may be undesirable in some settings. Transverse/oblique orientation of the source and detector frames are also contemplated.

Ideally the second set of images are obtained at image angles covering an arc α of no more than about 120 degrees, preferably no more than about 100 degrees, and more preferably no more than about 80 degrees in the plane of the source-detector pair. Location of the source detector pair 110A, 120A toward the end of the arc is shown in FIG. 15B, where the source 110A is located toward the bottom of the source frame 410 and detector 120A is located toward the top of detector frame 450 just prior to completing a complete pass of the source and detector frame to acquire the second set of images. Thus, the arc shown in FIG. 15B is approaching α=45 degrees with still more travel along the frame 410 to be completed.

In other embodiments, the second set of images may be obtained at image angles covering an arc α of no more than about 180 degrees, about 160 degrees, about 140 degrees, about 120 degrees, about 100 degrees, and about 80 degrees in the plane of the source-detector pair. It is possible to acquire a maximum image angle of 180 degrees (plus or minus) when the source-detector pair for obtaining the second set of images are located in an alternative dorso-ventral arrangement configured for imaging around the subject's body 210 (i.e., dorso-ventrally). This is similar to the present arrangement in CT scanners which include a C-arm that rotates around the subject's body 210 (e.g., relative to the spine of the subject 200) to acquire images. Thus, image angles covering an arc α of about 180 degrees are possible and in either direction (dorsal or ventral) depending on the desired imaging of the subject 200. In this arrangement, the source-detector pair may be on respective source and detector frames that are curved or arcuate and configured for rotated by the motion assembly 400 around the subject's body 210 (see e.g., embodiment of the imaging device of FIGS. 18 to 24 having the source unit 112 with a ring structure surrounding the subject's body).

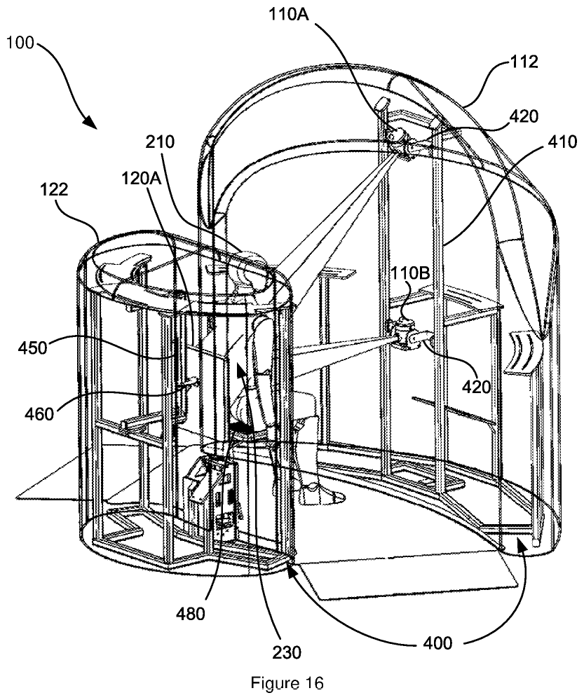

In some embodiments, it may be desirable to provide a supplementary source 110B on the source frame 410 as shown in FIG. 16. The linear and angular/rotational motion of the supplementary source 110B is also controlled by the controller 140 in a manner similar to that described for the source 110A and detector 120A. Advantageously, provision of the supplementary source 110B reduces the distance required to be travelled by source 110A to obtain the second set of images at each of the plurality of image angles. In this arrangement, it may be possible for control of the source 110A and supplementary source 110B to be coupled such that they move along the source frame 410 together, i.e. translating as one. In some embodiments, it is possible that operation of the sources 110A,B and detector 120A may be coordinated by the controller 140, such that a single detector 120A may be used to obtain the second set of images at each of the plurality of image angles. This typically involves the controller 140 controlling operation of the linear and angular actuators for source 110A and source 110B such that movement of the supplementary source 110B is in synchrony with the source 110A of the source-detector pair 110A, 120A. Furthermore, the controller 140 controls operation of the sources 110A,B and detector 120A to acquire images at each of the plurality of image angles required for the second set of images. In some arrangements, it may be preferable to also provide a supplementary detector 120B (not shown) on the detector frame 450 with the supplementary detector being operable under the control of the controller 140 in a manner similar to and typically in synchrony with the sources 110A,B and detector 120A. Thus, it may be possible for control of the detector 110B and supplementary detector 120B to be coupled such that they move along the detector frame 450 together, i.e. translating as one.

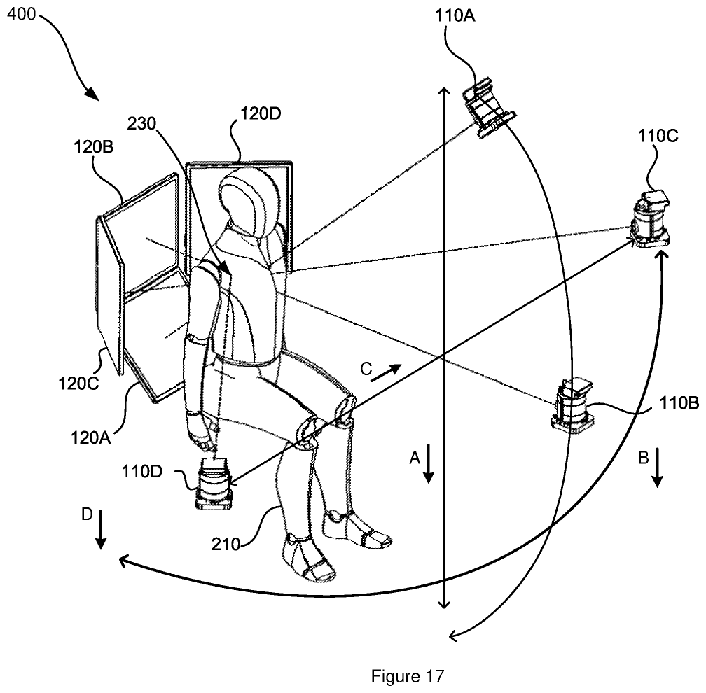

FIG. 17 is a schematic illustration showing various arrangements of the source-detector pairs that may be used to obtain the second set of images according to various embodiments, and with various features as described herein. For simplicity, the source and detector frames have been omitted to more easily illustrate the motion of the source-detector pairs that would be coupled to such frames in practice. Additionally, the energy beams emitted by the sources 110 towards the detectors 120 have been simplified to show a projection line through the subject 200. In one arrangement already described with reference to FIGS. 15A,B, as source 110A moves downward in direction A on a vertical source frame, detector 120A moves in synchrony upward on a vertical detector frame with rotational adjustments of the source 110A and detector 120A made along the way. Similarly the source 110A could equally move from a bottom position upward during acquisition of the images (with corresponding downward movement of the detector 120A). In another arrangement, the source 110B and corresponding detector 120B may be mounted on an arcuate source frame, and an arcuate detector frame which are also vertically oriented. As source 110B moves along the arcuate source frame in direction B, detector 120B moves in synchrony upward along the arcuate detector frame. Similarly, the source 110B and detector 120B could equally move in the opposite directions during acquisition of the images. Due to the arcuate frames, rotational adjustment will not be required as the source 110B and detector 120B translate along the frame. It is to be understood that in some embodiments the source frame may be arcuate with the detector frame being straight (or vice versa).

In another arrangement, a source 110C is mounted on a horizontal source frame and detector 120C is mounted on a horizontal detector frame. As source 110C moves along the frame in direction C, detector 120C moves in synchrony along the detector frame in the opposite direction with rotational adjustments of the source 110C and detector 120C made along the way. Similarly, the source 110C and detector 120C could equally move in the opposite directions during acquisition of the images (that is, in the same azimuthal direction in the horizontal plane of the source-detector pair). In another arrangement, the source 110D and corresponding detector 120D may be mounted on an arcuate source frame, and arcuate detector frame, which are horizontally oriented. As source 110D moves along the arcuate source frame in Direction D, detector 120D moves in synchrony along the arcuate detector frame. Similarly, the source 110D and detector 120D could equally move in the opposite directions during acquisition of the images. Due to the arcuate frames, rotational adjustment will not be required as the source 110D and detector 120D translate along the frame.

While the motion assembly in relation to FIGS. 15A,B and FIG. 16 provides for relative movement of the sources 110 and/or detectors 120 along frames to which they are mounted, it is to be understood that the sources 110 and detectors 120 may be fixedly coupled with the source frame 410 or detector frame 450 respectively, with the frames themselves moving to provide the relative movement of the motion assembly 400. In such arrangements it may be preferred that the source frame 410 and detector frame 450 are arcuate frames to limit the volume of space required for the imaging device 100.

In the embodiments described with reference to FIGS. 15A to 16, the two or more source-detector pairs are movable by the motion assembly 400 to obtain the second set of images. However, in another arrangement, motion assembly 400 may comprise a movable platform 480 such as a chair or a tray (see, for example the tray 480 on which subject 200 is positioned lying down as shown in the embodiments of the imaging device 100 as shown in FIGS. 18 to 24) which supports the subject 200 between the source-detector pairs, and operable by the controller 140 to control movement of the subject 200 relative to obtain the second set of images at the plurality of image angles. The movable platform 480 may be operable to rotate the seated subject 200 e.g. around a cranio-caudal axis between the sources 110 and the detectors 120 although rotation around a dorso-ventral axis (i.e. in a frontal plane) may be preferred for a subject 200 lying on a platform. In such embodiments, the source-detector pairs may remain stationary for acquisition of both the time series of images and the second set of images.

To assist with accurate linear and angular positioning of the sources 110 and detectors 120 as they translate along the source and detector frames 410, 450 (or to assist with accurate positioning of the platform 480 as it rotates the subject 200), the imaging device 100 may comprise one or more positional sensors configured to sense one or both of linear and angular position of one or more sources 110 and/or one or more detectors 120 (and/or the platform 480) of the device 100 for input to the controller 140. The positional sensors may comprise sensors configured to detect and monitor changes e.g. in acceleration and orientation and may include for example optic sensors, accelerometers, gyroscopes, or other electromagnetic sensors, or an encoder, stepper motor or ditch position sensor or the like to calculate position as would be appreciated by one of skill in the art.

While the motion assembly 400 has been described with reference to FIGS. 15A to 17 in the context of a seated subject 200, it is to be understood that the subject 200 may be standing or lying on a platform or tray 480, such as will be described with reference to the embodiments of FIGS. 18 to 24.

Furthermore, the embodiments described in relation to FIGS. 15A to 17 provide for synchronised movement of both the source 110 and detector 120 in a source detector pair. However, it is contemplated in some embodiments, such as those that may involve a subject 200 lying supine on a platform, or bed 480 (see FIGS. 18 to 24), that a source, optionally accompanied by a supplementary source, may be configured to move craniocaudally on a straight or arcuate frame located above the patient 200, with a detector located on a detector frame beneath and in close proximity to the subject, with the source and detector configured to translate along the frame (with angular adjustment as determined by the controller) in a manner described in relation to FIGS. 15A to 17. Additionally/alternatively, in some embodiments, a source, optionally accompanied by a supplementary source, may be configured to move dorso-ventrally on a straight or arcuate frame located above the patient 200, with a detector located on a detector frame beneath and in close proximity to the subject, with the source and detector configured to translate along the frame (with angular adjustment as determined by the controller (not shown). In either the craniocaudal or dorso-ventral arrangements, with a sufficiently long detector, only minimal movement of the detector beneath the patient may be required and in some arrangements where the detector is large enough to detect all emissions from the source/s as they move, no movement of the detector may be required at all.

Another embodiment of the imaging device 100 is illustrated in relation to FIGS. 18 to 24. The same reference numerals have been used for corresponding features shown and described with reference to FIGS. 15 to 17, which also apply to the embodiments shown in FIGS. 18 to 24. The only exception is reference to a supplementary source 110B which in the embodiment of FIGS. 18 to 24 is referred to by numeral 110A.

The imaging device 100 of this embodiment of the disclosure comprises at least two energy sources 110 and at least two detectors 120 for detecting energy from the at least two energy sources 110 passing through the region 230 of the subject's body 210 located between the energy sources 110 and the energy detectors 120. A first source-detector pair is located in a first plane, and a second source-detector pair is located in a second plane. The first and the second plane intersect through the region 230 of the subject's body 210 to be imaged. In these figures, four pairs of energy sources 110 and detectors 120 are illustrated, although only two pairs need to be provided. The embodiments illustrated do include four pairs of energy sources 110 and detectors 120, and there may be provided two source-detector pairs in the first plane (e.g., 110A-120A, 110B-120B) and two source-detector pairs in the second plane (e.g., 110C-120C, 110D-120D).

The imaging device 100 as shown in FIGS. 18 to 24 further comprises an auxiliary energy detector denoted as 120E for detecting energy from one of the energy sources 110 providing a source-auxiliary detector pair. The motion assembly 400 of the imaging device 100 is configured to achieve relative movement between the source-auxiliary detector pair and the subject 200. The controller 140 is configured to operate the energy sources 110 and detectors 120 while stationary, to acquire a time series of in vivo images of the region 230 of the subject's body 210. The controller 140 is also configured to operate the motion assembly 400 and the source-auxiliary detector pair to achieve relative movement between the source-auxiliary detector pair and the subject 200 to obtain a second set of images at each of a plurality of image angles in a plane of the source-auxiliary detector pair. The plane of the source-auxiliary detector pair may pass through the region of the subject's body to be imaged. Thus, the source-auxiliary detector pair may be oriented such that the emitted energy beam can be received by the auxiliary detector through the region of the subject's body to be imaged.

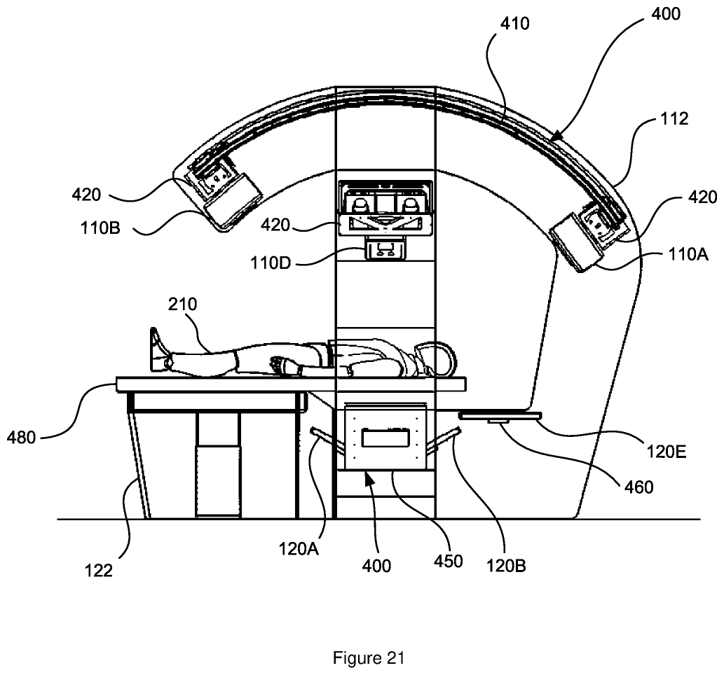

Many of the features above have been described with respect to the imaging device 100 shown in FIGS. 15 to 17. However, the main difference with this embodiment is that the imaging device 100 comprises an auxiliary energy detector 120E which is used to provide the second set of images, e.g., a set of images for construction of a geometric structure image of the region 230 of the subject's body 210. Significantly, the auxiliary detector 120E is not orthogonal to the axis of the beam produced by the corresponding energy source e.g., 110B as shown in FIGS. 21 to 24. The auxiliary detector 120E is positioned parallel to the subject's body and may be movable along a horizontal detector frame. Nonetheless, the auxiliary detector 120E is positionable relative to the corresponding source, e.g., via the motor assembly 400, such that images are able to be acquired of sufficient quality for structural imaging of the region of the subject's body.

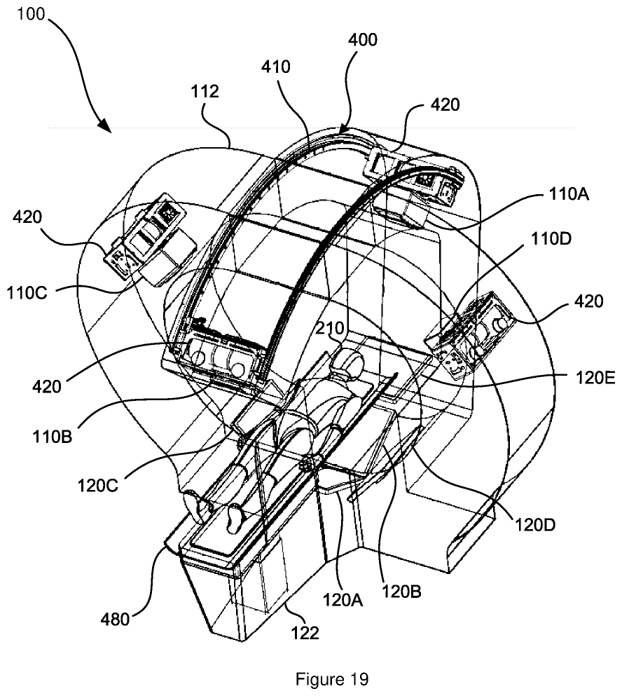

FIGS. 18 and 19 are perspective views of the imaging device 100 having the auxiliary energy detector 120E. In these embodiments, the subject 200 is positioned on a tray 480 in a supine position. The imaging device 100 includes a source unit 112 which includes a ring structure which supports source units 110C, 110D and an arm structure which supports source units 110A, 110B above the subject 200. The ring structure may be non-circular as shown, such as having an elliptical, ovular or other non-circular opening, however the ring structure is not limited to these arrangements and may be circular. The ring structure and the arm structure of the source unit 112 intersect and are perpendicular to one another in this embodiment although are not limited to this arrangement. Thus, the sources 110A, 110B, 110C and 110D are provided in a plus-sign or cross-sign spatial arrangement with sources 110A, 110B being in a first plane and sources 110C, 110D being in a second plane.

The arm structure of the source unit 112 illustrates a motor assembly 400 which includes coupling devices 420 for coupling the sources 110A, 110B to a curved or arcuate source frame 410. The source 110B is slidable along a rail of the source frame 410 above the subject's body 210 such that it can be positioned anywhere along the rail between the source 110A or at the furthest point of the arm structure of the source unit 112 (see also FIGS. 21 to 24). The coupling device 420 and motor assembly 400 operation may be similar to that described above in relation to FIGS. 15 to 17. The ring structure of the source unit 112 may also include a motor assembly 400 having coupling devices 420 as shown for source units 110C, 110D with an arcuate or curved source frame 410 (not shown) on which the source units 110C, 110D are translatable.

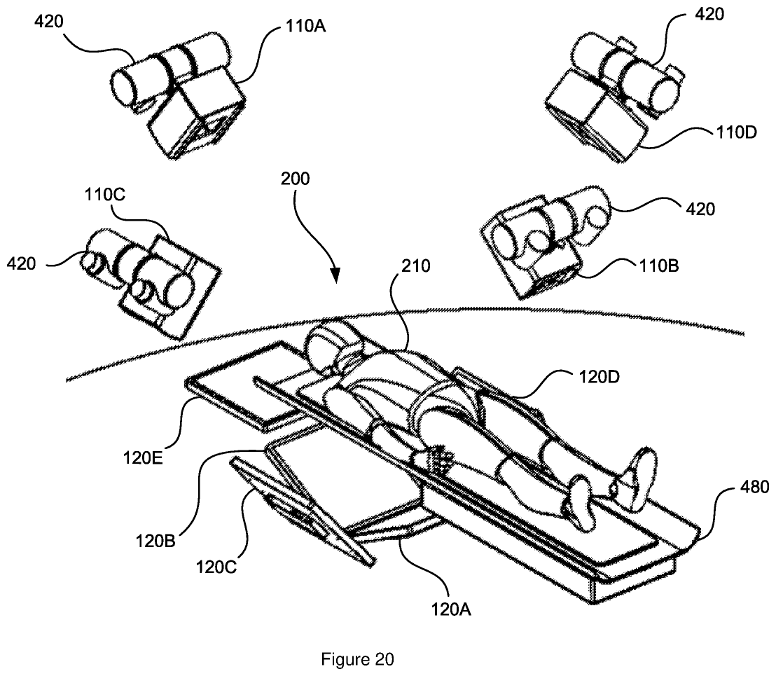

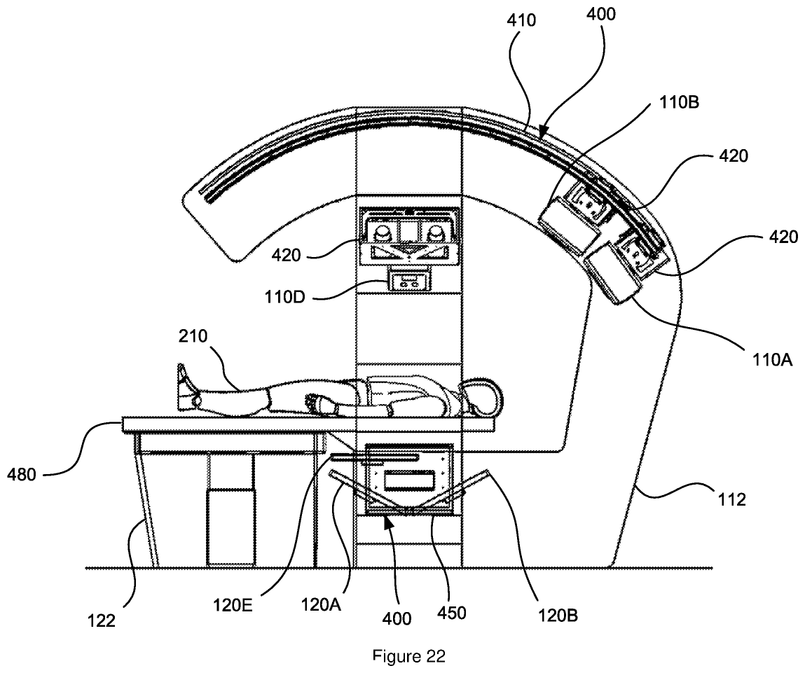

The imaging device 100 also includes a detector unit 122 which is located underneath the tray 480 on which the subject 200 is positioned. The detector unit 122 houses four detectors 120A, 120B, 120C, 120D and auxiliary detector 120E (see FIG. 20). The detector unit 122 may also include a motor assembly 400 (see also FIGS. 22 to 24) which includes a detector frame 450 and coupling devices 460 for the detectors 120A-E. The motor assembly 400 may include one or more detector frames 450 to allow for coupling of the detectors 120A-E via coupling devices 460 thereon (not shown). The detector frames may be straight or curved in shape. Although the embodiments illustrated include four pairs of source-detectors, the imaging device 100 may only include two pairs of source-detectors, and the auxiliary detector 120E. Furthermore, the embodiments illustrated include arcuate or curved source frames 410 and optionally detector frames 450, however this need not be provided and the frames 410 may be straight, for example. However, the curvature is advantageous to minimise the area occupied by the imaging device 100, in particular allowing a compact arrangement of detectors 120 to be provided in the detector unit 122.

The arrangement of the sources 110 and detectors 120 of the imaging device 100 are best shown in the perspective view of FIG. 20. FIG. 20 illustrates the same view as FIG. 18, however the exemplary detector unit 122 and source unit 112 are excluded, together with the motor assembly 400 for clarity. It is possible to observe the four sources 110A, 110B, 110C, 110D spatially arranged above the subject's body 210, and the four detectors 120A, 120B, 120C, 120D spatially arranged below the subject's body 210 positioned in the tray 480. The auxiliary detector 120E is currently positioned away from the subject's body 210 and is in a disabled configuration. The auxiliary detector 120E adopts this configuration when not in use, i.e., dynamic imaging is being performed using the pairs of source-detectors to acquire the time-series of images.

FIG. 21 is a side transparent view of the imaging device of FIGS. 18 and 19 and illustrates more clearly the auxiliary detector 120E in the disabled configuration. Also shown in this figure is the detail of the motor assembly 400 with detector frame 450 and a detector coupling 460 on the auxiliary detector 120E. Much of the motor assembly 400 has been omitted from this figure but it will be appreciated that the auxiliary detector 120E is able to translate parallel (or substantially parallel, or along a longitudinal axis relative) to the subject's body 210 along a detector frame 450 having a rail (similar to the source 110B on frame 410) to slot in below the tray 480 in the detector unit 122. Once translated to be positioned beneath the subject's body 210, the auxiliary detector 120E adopts the enabled configuration for use in providing the second set of images, e.g., the structural images of the region of interest 230.

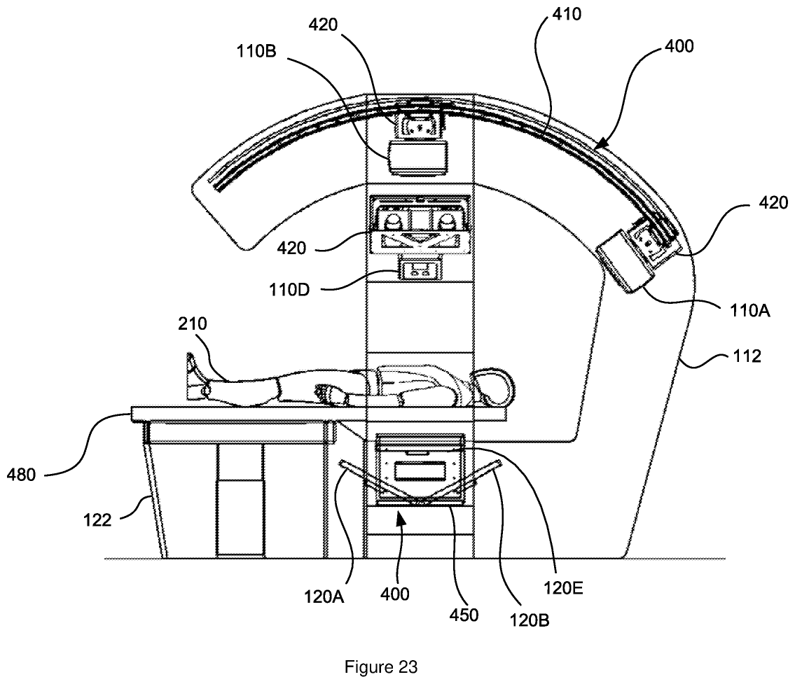

FIGS. 22 to 24 are transparent side views of the imaging device 100 shown in FIG. 21 with the auxiliary detector 120E shown in three different positions in an enabled configuration for obtaining the second set of images. The auxiliary detector 120E is moveable parallel to the subject's body 210 on the tray 480 via the motor assembly 400. In FIG. 22, the auxiliary detector 120E is located above the detector 120A and adopts a location centrally aligned with the subject's pelvis. In FIG. 23, the detector 120E has been moved centrally above the detectors 120A, 120B and adopts a location centrally aligned with the subject's chest. Finally, the detector 120E has been moved to be positioned above the detector 120B and adopts a location centrally aligned with the subject's neck. Advantageously, the movement of the auxiliary detector 120E is synchronised with the source unit 110B above which moves along the curved frame 410 of the motor assembly 400. This may occur via the controller 140 operating the motor assembly 400 and the auxiliary detector 120E-source 110B pair for coordinated relative movement, similar to that described above in relation to FIGS. 15 to 17. Thus, the controller 140 may operate the motor assembly 400 to allow for synchronised movement of both the source 110B and auxiliary detector 120E along their respective frames 410, 450 to acquire the second set of images.

In some embodiments, it may be desirable to provide a supplementary source 110A on the source frame 410 as shown in FIGS. 18 to 24. The linear and angular/rotational motion of the supplementary source 110A is also controlled by the controller 140 in a manner similar to that described for the source 110B and the auxiliary detector 120E. Advantageously, provision of the supplementary source 110A reduces the distance required to be travelled by source 110B to obtain the second set of images at each of the plurality of image angles. In this arrangement, it may be possible for control of the source 110B and supplementary source 110A to be coupled such that they move along the source frame 410 together, i.e. translating as one. In some embodiments, it is possible that operation of the sources 110A,B and auxiliary detector 120E may be coordinated by the controller 140, such that a single detector 120E may be used to obtain the second set of images at each of the plurality of image angles. This typically involves the controller 140 controlling operation of the linear and angular actuators for source 110A and source 110B such that movement of the supplementary source 110A is in synchrony with the source 110B of the source-auxiliary detector pair 110B,120E. Furthermore, the controller 140 controls operation of the sources 110A,B and detector 120E to acquire images at each of the plurality of image angles required for the second set of images.

In some arrangements, it may be preferable to also provide a supplementary detector 120 (e.g., any one of detectors 120A, 120B, 120C, 120D) (not shown) on the detector frame 450 with the supplementary detector 120 being operable under the control of the controller 140 in a manner similar to and typically in synchrony with the sources 110A,B and auxiliary detector 120E. Thus, it may be possible for control of the auxiliary detector 120E and supplementary detector 120 to be coupled such that they move along the detector frame 450 together, e.g. translating or rotating as one.

While the motion assembly 400 in relation to FIGS. 18 to 24 provide for relative movement of the sources 110 and/or detectors 120 along frames to which they are mounted, it is to be understood that the sources 110 and detectors 120 may be fixedly coupled with the source frame 410 or detector frame 450 respectively, with the frames themselves moving to provide the relative movement of the motion assembly 400. In such arrangements it may be preferred that the source frame 410 and detector frame 450 are arcuate frames to limit the volume of space required for the imaging device 100.

The above two aspects of the disclosure relate to imaging devices 100 which provide for obtaining a second set of images (e.g., able to be processed to provide the geometric structure, ideally three-dimensional structure, of the region of interest 230), that either use one of the detectors 120 from a pair of source-detectors in a plane (FIGS. 15 to 17) or provide an auxiliary detector 120E for use with a source 110B, which is beneficially configurable to be disabled or enabled during the imaging (FIGS. 18 to 24).

In some embodiments, the imaging device 100 of FIGS. 15 to 17 could optionally include the auxiliary energy detector 120E (not shown) for detecting energy from one of the energy sources 110 (e.g., any one of sources 110A, 110B, 110C and 110D) providing an source-auxiliary detector pair as described with reference to FIGS. 18 to 24. The motion assembly 400 may be further configured to achieve relative movement between movement between the source-auxiliary detector pair and the subject 200 for obtaining the second set of images at each of the plurality of images angles in a plane of the source-auxiliary detector pair, as described with reference to FIGS. 18 to 24.

In some embodiments, the imaging device 100 of FIGS. 15 to 17 could optionally include the at least one source-detector pair and the source-auxiliary detector pair being moveable by the motion assembly 400 to obtain the second set of images. Thus, two or three sets of source-detector pairs (e.g., including the source-auxiliary detector pair) may be used to obtain the second set of images.

In another embodiment, an imaging device 100 may be provided similar to FIGS. 15 to 17. However, any one of the detectors 120 may be used with any one of the sources 110 in obtaining the second set of images (not shown). That is, the disclosure is not limited to use of one of the defined source-detector pairs within a plane. Thus, a separate auxiliary detector 120E does not need to be provided and any of the detectors 120 may be operated with the sources 110 for providing the second set of images. In this embodiment, the motor assembly 400 may provide for operation with any combination of sources 110 and detectors 120 in the spatial arrangement, e.g., based on operation of the controller 140 being configured to control linear and/or angular motion of the source(s) 110 along the source frame 410 and the detector(s) 120 along the detector frame 450. The controller 140 may also be programmed to enable synchronous movement along of the source(s) 110 and detector(s) 120 along the respective source and detector frames 410, 450 in order to acquire the images.

The imaging device 100 of this embodiment of the disclosure comprises at least two energy sources 110 and at least two energy detectors 120 for detecting energy from the at least two energy sources 110 passing through the region 230 of the subject's body 210 located between the energy sources 110 and the energy detectors 120. A first source-detector pair is located in a first plane, and a second source-detector pair is located in a second plane, where the first plane and the second plane intersect through the region 230 of the subject's body 210 to be imaged. A motion assembly 400 is configured to achieve relative movement between at least one source-detector pair comprising at least one of the energy sources 110 and energy detectors 120, and the subject 200. A controller 140 is configured to operate the energy sources 110 and detectors 120 while stationary, to acquire a time series of in vivo images of the region 230 of the subject's body 210. The controller 140 is also configured to operate the motion assembly 400 and the at least one source-detector pair to achieve relative movement between the at least one source-detector pair and the subject 200 to obtain a second set of images at each of a plurality of image angles in a plane of the source-detector pair.

Although not shown, the imaging device 100 of this embodiment of the disclosure may have similar features to that shown in FIGS. 15 to 17 (omitting the auxiliary detector 120E) or FIGS. 18 to 24 (optionally including the auxiliary detector 120E, although not required). In some embodiments, the imaging device 100 of this embodiment may include a detector 120 that is configurable to move in position below a movable platform 480 (e.g., a tray, bed or chair) on which the subject 200 is positioned, similar to location and operation of the auxiliary detector 120E, via the detector frame 450 of the motor assembly 400. In this example, the detector unit 122 may include a motion assembly 400 having a detector frame 450 on which one of the detectors 120 may be translatable and/or rotatable from an angled position, e.g., see detectors 120A, 120B, 120C and 120D spatially arranged in FIG. 20, to a planar position relative to the subject's body 210, see e.g., location of detector 120E. For example, the detector 120B (although any of the detectors 120 may be movable) may be translatable from beneath the movable platform or tray 480 to the position of the detector 120E shown in FIG. 20. More preferably, the detector 120B may translate via the motion assembly 400 on the detector frame 450 being operated by the controller 140 to a position in any one of FIGS. 22 to 24, in the enabled configuration for acquiring the second set of images. Thus, the auxiliary detector 120E may not be required. Any one of the detectors 120 may be translatable and/or rotatable from their respective angled positions to the position of the auxiliary detector 120E as shown in FIGS. 18 to 24.

Thus, the detector 120B (or any of the detectors which are translatable and/or rotatable) may form a source-detector pair with any one of the sources (110A, 110B, 110C and 110D) for obtaining the second set of images. The imaging device 100 is not limited to obtaining the second images using one of the defined source-detector pairs within the first or second plane. The motion assembly 400 advantageously provides the means through the detector frame 450 and couplings 460 to allow for one or more detectors 120 to be translated, e.g., linearly and/or angularly, for receiving energy emitted from any one of the sources 110.

The imaging device 100 may optionally include the auxiliary energy detector 120E (not shown) for detecting energy from one of the energy sources 110 (e.g., any one of sources 110A, 110B, 110C and 110D) providing an source-auxiliary detector pair as described with reference to FIGS. 18 to 24. The motion assembly 400 may be further configured to achieve relative movement between movement between the source-auxiliary detector pair and the subject 200 for obtaining the second set of images at each of the plurality of images angles in a plane of the source-auxiliary detector pair, as described with reference to FIGS. 18 to 24.

In some embodiments, the imaging device 100 could optionally include the at least one source-detector pair and the source-auxiliary detector pair being moveable by the motion assembly 400 to obtain the second set of images. Thus, two or three sets of source-detector pairs (e.g., including the source-auxiliary detector pair) may be used to obtain the second set of images.

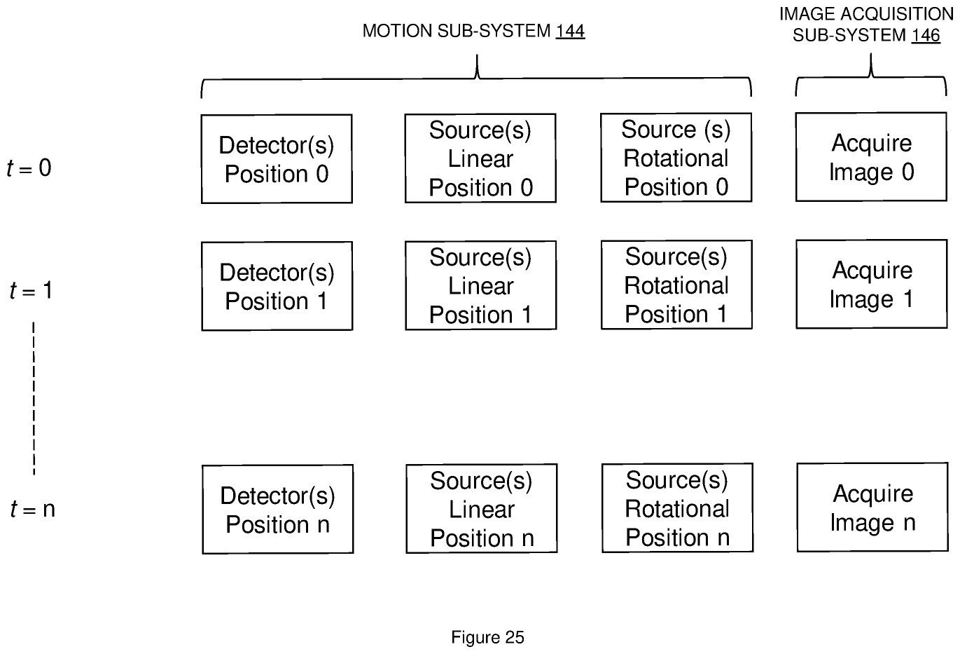

Ideally the second set of images are obtained at image angles covering an arc α of no more than about 120 degrees, preferably no more than about 100 degrees, and more preferably no more than about 80 degrees in the plane of the source-auxiliary detector pair and/or the at least one source-detector pair (similar to that shown in FIG. 15B. In other embodiments, the second set of images may be obtained at image angles covering an arc α of no more than about 180 degrees, about 160 degrees, about 140 degrees, about 120 degrees, about 100 degrees, and about 80 degrees in the plane of the source-detector pair. It is possible to acquire a maximum image angle of 180 degrees (plus or minus) when the source-detector pair for obtaining the second set of images are located in an alternative dorso-ventral arrangement configured for imaging around the subject's body 210 (i.e., dorso-ventrally). Thus, image angles covering an arc α of about 180 degrees are possible and in either direction (dorsal or ventral) depending on the desired imaging of the subject 200 (see e.g., embodiment of the imaging device of FIGS. 18 to 24 having the source unit 112 with the ring structure). FIG. 25 is a schematic timing diagram illustrating operation of the source 110 and detector 120 to acquire a series of n images in the second set of images. At t=0 the controller 140 positions the detector 120 at Position 0 and positions source 110 at linear Position 0 and angular Position 0 before actuating the source 110 and detector 120 to acquire Image 0. The controller 140 then adjusts the detector 120 to Position 1 and the source 110 to Linear Position 1 and Angular Position 1 and actuates the source 110 and detector 120 to acquire Image 1. This is repeated in sequence until n images at the required plurality of image angles have been obtained, forming the second set of images. While the angular position of the detector 120 is not altered in FIG. 25, it is to be understood that angular position could be altered in some embodiments.

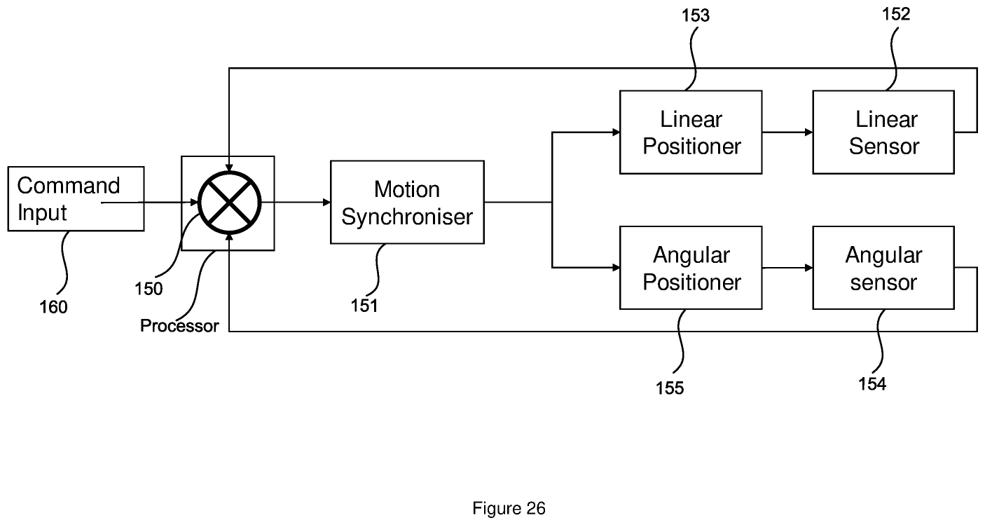

Ideally controller 140 is configured with a motion control module for obtaining the second set of images, as represented schematically in FIG. 26. Ideally, the controller 140 is preprogramed with or receives one or more operational parameter inputs via an input command 160 to the controller 140, indicating the arc over which the plurality of images in the second set is to be acquired. In some embodiments, the controller 140 may also receive as an operational parameter input the number of images to be obtained in the second set although this may be a fixed operational parameter of the device 100 which may be programmed to acquire e.g. fewer than about 20 images per 10 degrees of arc motion, or fewer than about 15 images per 10 degrees of arc motion, or fewer than about 12 images per 10 degrees of arc motion, or fewer than about 10 images per 10 degrees of arc motion. Thus, controller 140 may configured in certain embodiments to operate the at least one source-detector pair or the source-auxiliary detector pair (embodiments of FIGS. 18 to 24) and the motion assembly 400 to obtain the second set of images such that it acquires fewer than about 200 images, or fewer than about 120 images, such as fewer than about 100 images, or fewer than about 80 images possibly fewer than about 50 images, or fewer than about 40 images, such as about or fewer than about 20 images, such as about 10 images.

Unlike images obtained in standard tomography, the images obtained in the second set are not required to provide diagnostic grade resolution when reconstructed into a geometric structure image. Typically the second set of images are obtained at image angles covering an arc of no more than about 120 degrees, preferably no more than about 100 degrees, and more preferably no more than about 80 degrees in the plane of the source-detector pair or the source-auxiliary detector pair. In order embodiments, such as where dorso-ventral imaging is possible, the second set of images may be obtained at image angles covering an arc of no more than about 180 degrees, no more than about 160 degrees, no more than about 140 degrees, no more than about 120 degrees, no more than about 100 degrees, and no more than about 80 degrees in the plane of the source-detector pair or the source-auxiliary detector pair. In some embodiments, controller 140 is programmed to calculate the angular separation of each of the plurality of images based on the received operational parameters, and thus the required position and angle of each source 110 and detector 120 used to generate the second set of images.

Controller 140 is also configured to receive an input command 160 to initiate acquisition of the second set of images. The input command 160 is processed by processor 150 having a motion synchroniser 151 configured to coordinate operation of the linear positioner/actuator 153 and angular positioner/actuator 155 of each source 110 and detector 120 being moved. Data from positional sensors including linear sensor 154 and angular sensor 156 may also be used by processor 150 to determine control signals to send to motion synchroniser 151 to achieve movement of the one or more sources 110 and one or more detectors 120.

The controller 140 may be configured to operate the motion assembly 400 (e.g. the platform 480 and/or the actuators/positioners 153, 156 moving the one or more sensors 110 and detectors 120 for continuous relative movement while the source-detector pair or the source-auxiliary detector pair (embodiments of FIGS. 18 to 24) obtains the second set of images at each of plurality of image angles. That is, for embodiments where the source/s 110 and/or detector/s 120 are on frames, the movement of the source 110 or detector 120 on the respective frame may be continuous, with activation of the source and detector to achieve image acquisition when the controller 140 determines that they have moved to the correct position to obtain the required image angle. Alternatively, the movement may be discrete, with the controller pausing movement while the image is acquired at each of the plurality of angles required for the second set of images.

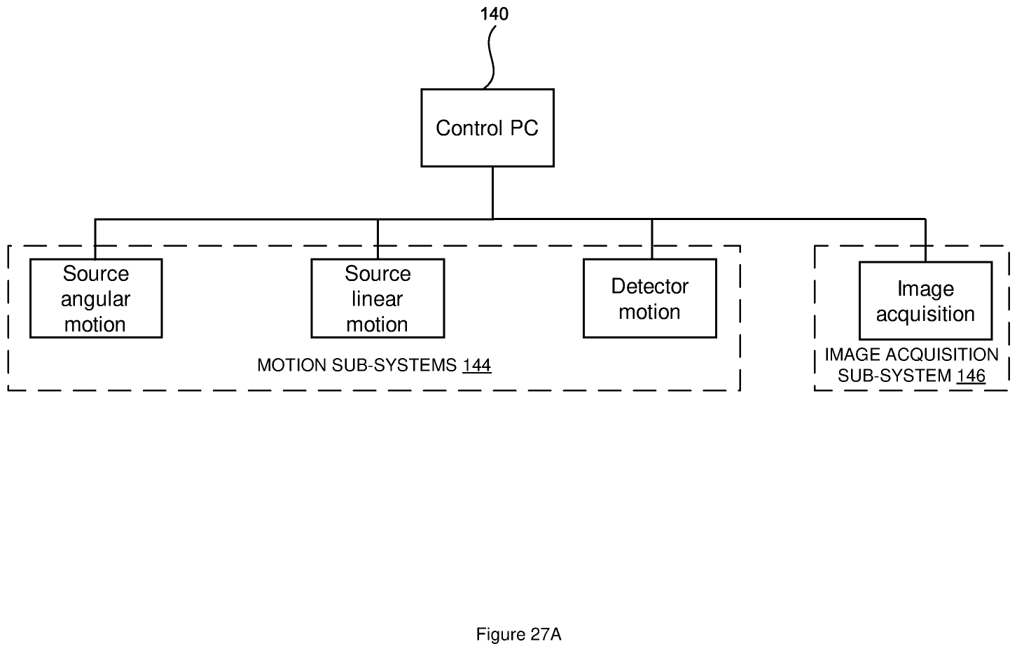

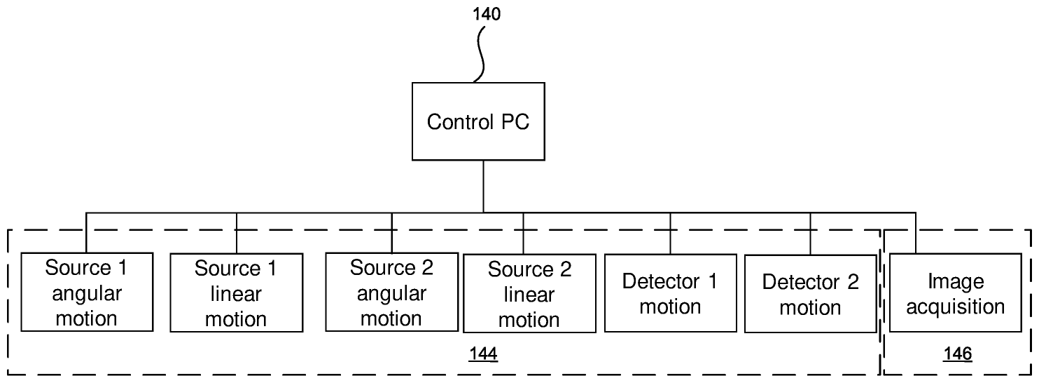

FIGS. 27A and 27B are schematic illustrations of control system architectures that may be utilised in various embodiments disclosed herein for acquisition of the second set of images. In FIG. 27A, controller 140 controls operation of an imaging device 100 having a single source-detector pair or the source-auxiliary detector pair (embodiments of FIGS. 18 to 24) with a motion subsystem 144 providing control over the angular and linear motion of the source 110 as well as the position of the detector 120. Image acquisition subsystem 146 controls operation of the source and detectors for the acquisition of images, when the motion subsystem 144 has suitably positioned the source 110 and detector 120 for the required image angle. FIG. 20B illustrates corresponding architecture for an imaging device 100 utilising two source-detector pairs or the source-auxiliary detector pair (embodiments of FIGS. 18 to 24) and at least one source-detector pair for the acquisition of images at the plurality of image angles required for the second set of images. In FIG. 27B, motion subsystem 144 provides control over the angular and linear motion of two sources (Source 1, Source 2) and two detectors (Detector 1, Detector 2). Image acquisition subsystem 146 controls operation of the sources and detectors for the acquisition of images, when the motion subsystem 144 has suitably positioned the sources and detectors for the required image angles. Relevantly, when both sources are located in a common plane the controller 140 may control operation of the source-detector pairs or the source-auxiliary detector pair (embodiments of FIGS. 18 to 24) and at least one source-detector pair to each acquire only half of the images required to complete the second set of images. In certain embodiments, this may half the image acquisition time.

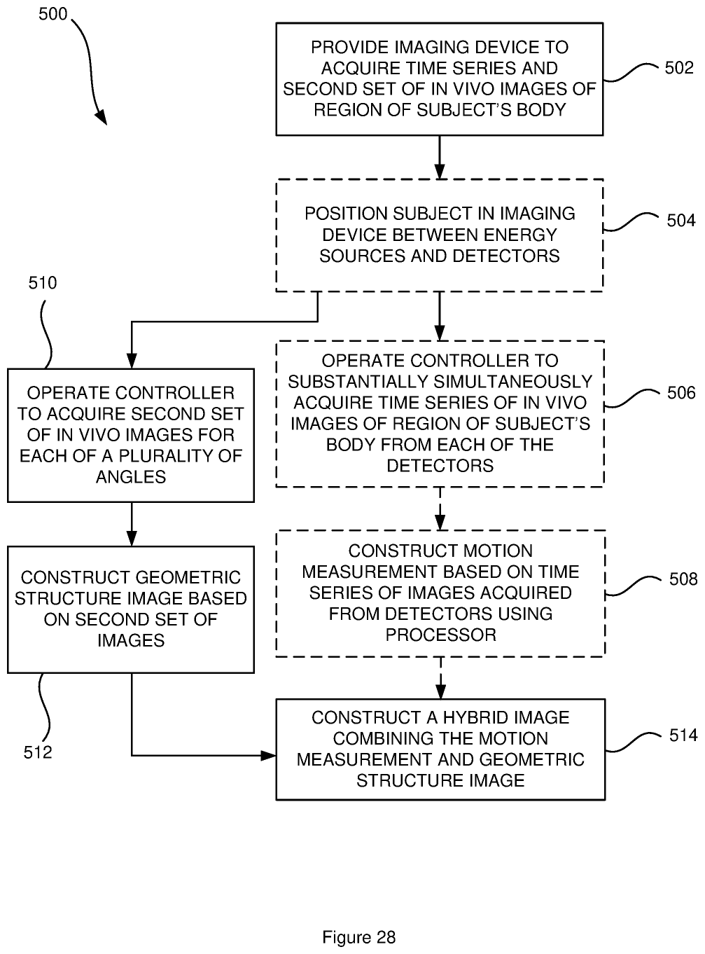

FIG. 28 is a flow chart showing steps in a method 500 for acquiring in vivo images of a region 230 of a subject's body 210 according to embodiments of the present disclosure. The method 500 includes in a step 502, providing an imaging device 100 according to any one of the embodiments disclosed herein and in a preferred step 504, positioning a subject 200 in the imaging device 100. The subject 200 may be positioned in the imaging device 100 in an upright seated or standing position, or lying e.g. supine on a tray, table or platform 480 between the sources 110 and detectors 120. In a step 506 the controller 140 is operated to acquire the time series of in vivo images of the region of the subject's body 210 simultaneously or substantially at the same time from each of the detectors 120 and in a step 508 a motion measurement is constructed by a processor 150 based on the time series of images acquired from each of the detectors 120. In a step 510 the controller 140 is operated to acquire the second set of in vivo images of the region 230 of the subject's body 210 for each of the plurality of angles including by operating the motion assembly 400 and in a step 512 a geometric structure image is constructed by a processor, based on the second set of images obtained from the detectors 120 for each of the plurality of angles. In a step 514 a hybrid image is constructed in which the motion measurement and the geometric image are combined.

FIG. 13 is a schematic diagram of components of a source unit 112 and detector unit 122 of the imaging device 100 according to some embodiments of the disclosure. The detector unit 122 and source unit 112 are shown in broken lines to indicate that this is an exemplary arrangement of the components and systems of the imaging device 100, which may vary as would be understood by the skilled addressee. For example, the XV processing unit 186 (optionally provided in the detector unit 122) may instead be located in the source unit 112. Alternatively, the XV processing unit 186 may not be included in the imaging device 100 and may instead be provided via a cloud-based server having the XV processing application for off-board processing of the image data. Moreover, in some embodiments, the control system 152, the safety system 182, the output device 117 and the communication system 188 of the source unit 112 may instead be located in the detector unit 122. In some embodiments, components such as the control system 152, the output device 117, the safety system 182, and the communication system 188 may be located outside of the source and detector units entirely, such as in a control room or other location distinct from the apparatus containing the sources, detectors and generators. It is to be understood that various locations and arrangements between these components are contemplated and are within the scope of this disclosure.

The processor 150 and processing unit 186 of FIG. 13 used to implement certain steps of the methods 300, 500 of embodiments of the disclosure may include a micro-processor configured to receive data from components of the device 100 or a computing server, such as through a wireless or hard-wired connection (not shown). The controller 140 may comprise any suitable computing device and may include a programmable logic controller (PLC) and/or an embedded PCB (not shown) in some embodiments. The controller 140 may contain or store a number of predefined protocols or steps in a non-volatile memory such as a hard drive. Protocols may be programmable by the operator of the imaging device 100 to implement a number of steps for the method 300 as performed by the processors 150 and 186, or they may be predefined. Additionally/alternatively, the controller 140 and processors 150 and 186 may include any other suitable processor or controller device known to a person skilled in the art. The steps performed by the processors 150 and 186 may be implemented through a controller 140 and further in software, firmware and/or hardware in a variety of manners as would be understood by a person skilled in the art.

FIG. 13 also excludes some additional components and systems which would form part of the imaging device 100 to simplify the diagram. For example, the imaging device 100 may include one or more memory devices (not shown) in order to store various types of data including image data and prior-acquired patient data, and also software instructions for performing image acquisition processing workflows and XV processing, as will be described in more detail. The schematic diagram of FIG. 13 also omits some of the internal bus lines between various components and systems for simplicity. The excluded aspects would be readily appreciated by a person skilled in the art who would be able to readily supply the omitted software, firmware and/or hardware.

The source unit 112 includes one or more energy sources 110 (ideally at least two, or three energy sources denoted as 110A, 110B) which are powered by one or more source generators 114 forming part of a power supply 184 for the imaging device 100. In other embodiments (not shown), the one or more source generators 114 forming part of the power supply 184 may be located externally to the source unit 112 (and to the detector unit 122 in some embodiments) of the imaging device 100. A control system 152 having the controller 140 and processor 150 may be configured to operate the energy sources 110 and detectors 120 of the detector unit 122 for scanning the region 230 of the subject's body 210. In other embodiments (not shown), the processor 150 of the control system 152 may be located externally to the source unit 112 of the imaging device 100 (and to the detector unit 122 in some embodiments), such as to allow for off-board processing of the image data. The source unit 112 may also include a safety system 182 in communication with the control system 152. The safety system 182 may include an emergency stop 180 in the form of a software or hardware component of the imaging device 100. The emergency stop 180 may be located on a surface of the source unit 112 adjacent the subject 200 (not shown). The emergency stop 180 may include an actuator, such as a depressible button or switch, for powering off the imaging device 100 in the event of an emergency. If the emergency stop 180 is actuated, the controller 140 of control system 152 may be operable to stop acquisition of the images via the energy sources 110 and optionally, directly switching off power to the imaging device 100 via the power supply 184 (not shown), in order to prevent inadvertent generation of radiation or energy. In some embodiments the emergency stop function is built into the actuator that controls operation of the generators 114 which require a hand operated switch or foot pedal to be depressed in order for the generators to emit energy, and wherein operation of the generators 114 will cease when the pressure applied to the switch or pedal is removed.

The source unit 112 may also include one or more output devices 117 which may include a display 118 and a speaker 119 as shown in FIG. 13. A display 118 may be located on a surface of the source unit 112 (not shown) in the subject's line of sight when positioned in the scanner 100. Although not shown, the imaging device 100 may also include a speaker 119 positioned in the source unit 112 and/or the detector 122. The output device 117 is provided to enable communications to be delivered to and/or from the subject 200 and/or operator and the imaging device 100 via a communication system 188. For example, the control system 152 via the processor 150 may output instructions to the subject 200 and/or operator via the output device 117. The instructions may be provided on the display 118 and/or via the speaker 119.

As shown in FIG. 13, the detector unit 122 includes one or more detectors 120 (preferably at least two detectors 120A, 120B) operable by the controller 140 of the control system 152 for acquiring a time series of in vivo images and a second set of in vivo images of the region 230 of the subject's body 210. The time series of images may be used as an input to the XV processing unit 186, as previously described, for producing XV three-dimensional motion fields of the region 230 of the subject's body 210, such as the lungs or heart. The XV processing unit 186 may alternatively be provided off-board via a server or cloud-based system in some embodiments. The second set of images may be used as an input to the XV processing unit 186 for producing a geometric structure image, preferably a three dimensional geometric structure image of the region 230 of the subject's body i.e. the lungs or the heart, as represented in the three-dimensional motion fields.

In various embodiments described herein, all of the sources 110 may be located on one side of the imaging device 100, such as in front of the subject's body 210, and all of the detectors 120 may be located on an opposite side of the imaging device 100, such as behind the subject's body 210. The sources 110 may all be located within a first housing denoted as the source unit 112 and the detectors 120 may all be located within a second housing denoted as the detector unit 122 although it is to be understood that this is merely one arrangement, and multiple source units 112 and/or multiple detector units 122 may be provided in some embodiments. In such arrangement, there is sufficient space between the source unit 112 and detector unit 122 for the subject 200 to move in and out of the scanner 100 as the sources 110 and detectors 120 may extend circumferentially around the subject 200 at angles of substantially less than 180 degrees, such as only approximately 45 to 80 or 90 degrees. This advantageously enables access to the imaging device 100 for various patient groups, including young children, the elderly, and patients with language, hearing or cognitive impairment, who are unable to be readily scanned due to positioning issues within traditional scanners and/or the inability to follow instructions for the scanning to be completed.

The imaging device may be configured for use with one or more of x-ray imaging, ultrasound imaging, and magnetic resonance imaging (MRI). The x-ray imaging may include fluoroscopic imaging and/or computed tomographic x-ray velocity (CTXV) imaging.

The region of the subject's body to be imaged may include at least part of the lungs of the subject. The imaging device may image part of the lung or the whole lung. The imaging device may also image both lungs of the subject. Alternatively, the region to be imaged may include part or the whole of the heart or brain of the subject. The region to be imaged may include parts of the body other than organs, including tissues, such as abdominal tissues.

Advantageously, the present disclosure provides an imaging device and method that provides for acquisition of both a time series of images for obtaining a motion measurement of the region, and a second set of images for constructing a geometric structure image. These sets of images can be processed to produce a hybrid view of the region of interest, e.g. the lung, which provides functional information from the motion measurement, as well as anatomical context from the geometric structure image.

It is possible to supplement the motion measurements with geometric structure information obtained from e.g. a CT scan. However, there are several disadvantages associated with this, such as e.g. the requirement to relocate the subject from one apparatus to another, timing the availability of the two imaging apparatuses so that contemporaneous motion measurement images and CT images are obtained, and the relatively high dose of radiation to which the subject is exposed during acquisition of the CT image which typically involve hundreds or in some cases, thousands of image slices through the region of interest in order to produce 3D images of diagnostic quality. A further difficulty arises from the technical complexity of combining the images obtained for the motion measurements with the CT images. This is not a straightforward task since the two data sets from two different apparatuses need to be aligned in order for the combined images to be useful. This is further complicated by the fact that the subject has had to move between apparatuses and is unlikely to be in exactly the same position when the second set of images is obtained.

The present disclosure provides an elegant solution whereby it is possible to e.g. mount one of the source-detector pairs (optionally used to obtain the time series of in vivo images on a frame) or a source-auxiliary detector pair that permits relative movement between at least the source and the subject or both the source and the detector and the subject. This enables a single imaging system to obtain the time series of images (i.e. a first set of images) used for construction of the motion measurement as well as the second set of images used for construction of a geometric structure image. Additionally, various advantages concerning the compact form factor and lower dosages (relative to diagnostic CT) are attractive, particular to vulnerable subject such as children and babies.

It is to be understood that various modifications, additions and/or alternatives may be made to the parts previously described without departing from the ambit of the present invention as defined in the claims appended hereto.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that the following claims are provided by way of example only, and are not intended to limit the scope of what may be claimed in any future application. Features may be added to or omitted from the claims at a later date so as to further define or re-define the invention or inventions.

1. An imaging device for acquiring in vivo images of a region of a subject's body, the imaging device comprising:
at least two energy sources;
at least two energy detectors for detecting energy from the at least two energy sources passing through the region of the subject's body located between the energy sources and the energy detectors, wherein a first source-detector pair is located in a first plane, and a second source-detector pair is located in a second plane, wherein the first plane and the second plane intersect through the region of the subject's body to be imaged;
a motion assembly configured to achieve relative movement between at least one of the source-detector pairs and the subject; and
a controller configured to:
operate the energy sources and detectors while stationary, to acquire a time series of in vivo images of the region of the subject's body; and
operate the motion assembly and at least one source-detector pair to achieve relative movement between the at least one source-detector pair and the subject to obtain a second set of images at each of a plurality of image angles in the plane of the source-detector pair.

2. The imaging device according to claim 1, further comprising a third source-detector pair located in the first plane, and a fourth source-detector pair located in the second plane.

3. The imaging device according to claim 1, wherein the time series of in vivo images are used to generate a motion of the region and the second set of images are used to construct a geometric structure image of the region.

4. The imaging device according claim 1, wherein the second set of images are obtained at image angles covering an arc in the plane of the source-detector pair of no more than one of: about 180 degrees, about 160 degrees, about 140 degrees, about 120 degrees; about 100 degrees; or about 80 degrees.

5. The imaging device according to claim 1, wherein:
the controller is configured to operate the at least one source-detector pair and the motion assembly to obtain the second set of images such that it acquires less than one of: about 200 images; about 120 images; about 100 images; about 80 images; about 50 images; about 40 images; about 20 images; or about 10 images; and/or
the controller is configured to operate the motion assembly for continuous relative movement while the source-detector pair obtains the second set of images at each of the plurality of image angles.

6. (canceled)

7. (canceled)

8. The imaging device according to claim 1, wherein the motion assembly comprises a source frame supporting the source of the at least one source-detector pair, wherein the controller is configured to control linear motion of the source along the source frame to obtain the second set of images.

9. The imaging device according to claim 8, wherein the source frame is one of: a straight frame; or a curved frame.

10. The imaging device according to claim 1, wherein the motion assembly comprises a detector frame supporting the detector of the at least one source-detector pair, wherein the controller is configured to control linear motion of the detector along the detector frame to obtain the second set of images.

11. (canceled)

12. (canceled)

13. The imaging device according to claim 6, further comprising a supplementary source on the source frame, wherein the controller is configured to control linear motion of the supplementary source along the source frame to obtain the second set of images, wherein provision of the supplementary source reduces the distance required to be travelled by any source to obtain the second set of images at each of the plurality of image angles.

14. The imaging device according to claim 13, wherein the controller is configured to control movement of the supplementary source in synchrony with the source of the source-detector pair.

15. (canceled)

16. (canceled)

17. The imaging device according to claim 6, wherein the source frame provides one or more couplings for angular and linear motion of any source coupled to the source frame, and wherein the controller is configured to control angular and linear motion of any source coupled to the source frame.

18. (canceled)

19. (canceled)

20. The imaging device according to claim 1, wherein two or more source-detector pairs are movable by the motion assembly to obtain the second set of images.

21. (canceled)

22. The imaging device according to claim 1, wherein the motion assembly comprises a movable platform operable by the controller to control movement of the subject relative to the one or more source-detector pairs to obtain the second set of images.

23. The imaging device according to claim 22, wherein the movable platform is operable to rotate the subject around a cranio-caudal axis between the sources and the detectors.

24. The imaging device according to claim 22, wherein the movable platform is operable to rotate the subject around a dorso-ventral axis.

25. The imaging device according to claim 1, further comprising:
an auxiliary energy detector for detecting energy from one of the energy sources providing a source-auxiliary detector pair.

26. The imaging device according to claim 25, wherein the motion assembly is further configured to achieve relative movement between the source-auxiliary detector pair and the subject, and wherein the controller is further configured to operate the motion assembly and the source-auxiliary detector pair to achieve relative movement between the source-auxiliary detector pair and the subject to obtain the second set of images at each of the plurality of image angles in a plane of the source-auxiliary detector pair.

27.-51. (canceled)

52. A method for acquiring in vivo images of a region of a subject's body, the method comprising the steps of:
providing an imaging device according to any one of the preceding claims;
operating the controller to acquire the time series of in vivo images of the region of the subject's body simultaneously or substantially at the same time from each of the detectors;
operating the controller to acquire the second set of in vivo images of the region of the subject's body for each of the plurality of angles;
constructing, using a processor, a motion measurement based on the time series of images acquired from each of the detectors;
constructing, using a processor, a geometric structure image based on the second set of images obtained from the detectors for each of the plurality of angles; and
constructing, using a processor, a hybrid image in which the motion measurement and the geometric structure image are combined.

53. The method according to claim 52, comprising the step of, prior to operating the controller to acquire the images, positioning the subject in the imaging device between the energy sources and detectors.

54. The method according to claim 52, wherein:
the imaging device is configured for use with one or more of x-ray imaging, ultrasound imaging, and magnetic resonance imaging (MRI); and/or
the region of the subject's body to be imaged includes at least part of the lungs of the subject, and the hybrid image provides visible elements designating geometric features of the lungs.

55. (canceled)
